SEQUENCE LISTING (22 sequences)

SEQ ID NO: 1 - DNA, 1059 bp, Pyrococcus furiosus
atgagtaaaatcactgtaaaatctcctggtagggttaacttgatcggagagcacaccgattacacctatggttatgtaatgccaatggcaatcgatctctacacaataataacagccgaaaaatacgataaagtccagctatactccgaacacttcaacgaagagaaaacatttacattagataatctcacaaaggaaggatcatggatagactacgtaaaaggagtattgtgggttttaatccaagaagggtacaaaattggaggactaaaaggaaaaataacaggggaccttcccctcggagctggtctaagttcctcagcaagctttgaagttggaattttagaagtgctaaatcaactatataatttaaatatcgatccactcaaaaaggcattacttgcgaaaaaagctgaaaatgaatttgtgggagttccatgcggaatcttagatcaatttgcagttgtttttggaaaaaaggacaatgttatatttctagatacacaaacactgcaatatgaatatattccttttcccaaagacgtttcagtgttggtattttatactggagtaaagagagagttagcatcttcagaatacgcagaaagaaaacgcatagcagaagaaagtctgagaattcttgggaaagagtcctcaaaagaggtgacagaaaaggacttgggcaaacttcctcccctacacaggaagttcttcagctatatagttagagaaaacgctagagttcttgaagttagagatgcacttaaagagggggacatagaaaaggtaggaaaaattctcacaacagcacattgggatctagcggagaattatagagtcagctgcgaagagttagacttttttgttaaaaaagcgatggaattaggcgcttatggggccaggctaacaggcgctggatttggtggctcggcaatagcactagttgataaagataaggcaaaaacaataggagatgcaatattgagggaatatctcgcgaaattttcatggaaagctaaatatttcgtcgtaaaaccctccgatggtgtgggagtatga

SEQ ID NO: 2 - DNA, 1044 bp, Thermus thermophilus
atgggcttccaagaggtttacggcgtcctgccccaggccagcgcccaggcccccggccgggtgaacctcctgggggagcacacggactaccaggaaggctacgtcctccccaccccgatcccctacttcacccgggtggaggccgcccccctcgagggggcggtggaggccttcagcgagaacctgggggagctccgggcccgccccctctcctccccgccccaaggggacttcctggactacctcctcggggtggtccgggccctccgggaggccgggcacgaggtggcgggggccaggttctacgttcgcagcgacctccccatgggggcgggcctctccagctccgccgccctcgaggtggcggccctcagggccctccgcaccctctaccgcctccccttgagcgacctggaggtggcccgcctcgcccagaaagcggaggtggagtacgtgggggtccggtgcgggatcatggaccagatggcggcaagcctgggccagccggggcaggccctcttcctggacacccggaccctggcctacgagaaccttccccttcccccgggggtgcgggtggctgtcctggacctcgggcttgggcgcaggctggcggaggccgggtacaaccggcgccgccaggaggcggaggaggcggccaagaggctcggggtgcggtccctcagggacgtggccgacctctgcctggtggaaagcctcccttcgcccctggaccggcgggcccggcacgtggtgagcgagaaccttagggtcctccggggggtggaggccctaaggcggggggacgcccgggccttcggggagcttatgacgcaaagccaccgctccctcgcccaagactacgaggtgaacctgcccgagctggacgccctggtggaggaggccctgcgggccggggcctacggggccaagctcacgggggcgggcttcggcggggccgtggtggccctggtggccgaaagccgcttccccgccttcagggaggccctggcccggcgcttccccgacctcaaggtcctctaa

SEQ ID NO: 3 - DNA, 1167 bp, Streptococcus thermophilus
atgaatacatcacagttaagagaaaagtttaaagaagtttttggtgtagaagcagatcatactttcttttcaccaggtcgtattaatttgattggtgagcatacggactacaatggaggtaacgtccttccggtagctattaccctaggtacttacggagcggcccgcaaacgtgatgacaaagttttgcgtttcttctcagctaactttgaagagaagggaatcatcgaagtgccacttgaaaatcttcgttttgaaaaagaacacaactggacaaactatccaaaaggtgttcttcatttcttgcaagaagctgggcatacgattgattcaggtatggatatttacatctatggtaacattccaaacggatcaggcttgtcatcatcatcatctttggaattattgattggtgttattgttgaaaaactttatgacattaaattggaacgcctggacttggttaaaatcggaaaacaaacggaaaatgactttattggcgttaactctggtatcatggaccaattcgctattggtatgggagctgatcaatgtgcgatttacttggacacaaatactctaaagtatgacttggtaccccttgacctcaaggataatgtcgtagtcatcatgaacactaacaaacgtcgtgaattggctgattctaaatacaatgaacgtcgtgctgaatgtgaaacagcagtatctgaactacaagaaaaattagatatccaaactctcggtgaattagacttcttgacatttgacgcatacagctatttgattaaagatgaaaaccgtatcaaacgtgcacgccatgtagttcttgaaaatcaacgtacacttcaagctcgtaaagctcttgaagcaggagatttggaaggctttggacgccttatgaatgcttctcatgtgtcattggaatatgattacgaagttacaggtcttgaacttgatactttggcacacacagcttgggaacaagaaggagtattaggagcccgcatgacaggagctggtttcggtggatgtgccattgcacttgtaaacaaagacaaagttgaagacttcaaaaaagcagttggtcaacgctatgaagaagtcgttggttatgcaccaagcttctatattgccgaagtaactggtggttcacgagtacttgattaa

SEQ ID NO: 4 - DNA, 1263 bp, Pyrococcus furiosus
atgaaagctataatacttgccgctggaaaaggtgaaaggcttaggccactcactgatgataggccaaaggttgttctaaagatcgccaacaaaccaataattagctatgttcttgaaaaccttgacccatttgtagatgaatttatcatagttgttaagtacatgaaggagaaagttattgatttactcggtgatgaatttcgcggaaagccaataacttatgtggagcagggagaagaagagggaacagctgcagcagtgtattccgtaaaagagtttatagagtctaatgaagagtttttcgttgtcaatggcgatttatattttgaacccgatgcagtaaagggccttcttcatgttttcaaaaaagagaaaggagatgcaggcatcgttgtgaaagaatttgagaacctcagccaatatggaatggtagaggtagagaacggtaaggtgaaagggataatagaaaagccaggcaatgtcaaaggatatgcaaacctaggaatatacattttcaagtcagatgtctttgattacatagagaacaccgaaataagtgagaggggagaatatgagattacagacacgataaacctcatgataaaagatggcaaaagtgtggtttactacaactatgaaggattctggagcgatattggaaggccatgggacttacttgaagtgaatgaatacattctaaaaactaagctaaaacacgagataagaggagttgtcgaagagggagcaacaataatcccacctgtggaaattggagagggaacaatagtaagggcaggaagctacataatcgggcccgttaagatcggaaaaaactgtagaatcgggccgaactgctacataagaccatatacaagcataggagacaactgccacataggaaatgccgttgaagttaaaaattccataataatggataattcaaatgcccctcacttaaactatgttggggattcaataatcggggaaaacacaaatcttggggctggaacaataacagctaacttgagacacgacaagggaactatcaaggttgaagttaaaggaaagttagaggattcagggaggagaaagctcggtgcaataattgggcacaacgtaaaagtagggataaacgtcacaatttatccaggaagaaaaattggtagtaactctttcataggcccaggagttatagttgataagaacatcccacaaaacgttcttgttacagttaagcaggagaaaaaggtgattgagaaatga

SEQ ID NO: 5 - DNA, 1047 bp, Thermus thermophilus
atgaaagggctcatcctggccgcgggaagaggcacgaggctccgtcccctgacccacacccgccccaagcccgccatccgggtagcggggcggcccatcatccactacgccgtggagaacctcctggaggccggggtccgggagatcggggtcgtggtctccccggagacggagcgggacctgaaggtggccctcgagggctaccccgtccgctacgtccttcaggaggaaccccaggggctggcccacgcggtggacgtggcccgggacttcctgggggaaagccccttcgtcctctacctgggggacaacctcttccaaaagggcatccggcgcttcctggaggccttcaagcccggcgtgagcgccgtcatcgccctggtgcgggtggaagacccaaggcagttcggcgtggccgtcctggagggcaaccgggtggtgcggcttttggaaaagcccaaggatcccccctcggacctggcggtggccggggtctacgtcttctcccccgaggttttggaggtcgtccggggcctcaagccctccgcccggggagagtacgagatcaccgacgccatccagggcctcattgaccggggcaaaacggtggtgggcgtggaggtggagggctggtggaaggacacgggccgccaccaggacctcctggacgcgaaccggctcctcctcgaggagctcacccccaaggtggagggggacgtggtggagagccagctcacgggccgggtggtggtggagaagggggcgaaggtggtgcggagcaccgtgatcggccccgcccacatcggcgcgggcgccgtggtggaggaggccttcgtgggccccttcacctccgtggggccccgggcccgggtggtgcggtcggaggtggagtactccctcctggaggaggaggccgtggtggaggaggtccgcctccgcctccaggagtgcatcctcggcgtccgggccgaggtgaagagccgggacggcctcccccgggcccaccgcctcatcctcggggacctctcccaggtggagctggcctga

SEQ ID NO: 6 - DNA, 870 bp, Streptococcus thermophilus
atgaaaggtattattcttgcaggtggttcagggacacgtttgtatccactgacacgtgctgcatccaaacaactgatgccgatttacgataaaccaatgatttactacccattgtcaacgcttatgttggctggaatcaaggaaatcctcatcatttcaacatcacaggaccttccacgttttgaagaactccttggcgatggttcagagtttggtatttccctttcctacgctgtacaaccaagtccagatggcttggctcaagcctttatcataggcgaagacttcatcggtgatgacagtgttgctcttgtgctcggggacaacattttccatggcaatggtctttctgcaatgcttcaacgtgctgaagctaaggaaaaaggcgcaacagtctttggctaccaagttaaagacccagaacgttttggtgttgttgagtttgatgatgatatgaatgccatctcaatcgaagaaaaaccagaacatccaaaatcaaactttgctgtaacaggcctttatttttatgataatgatgtcgtggaaattgctaaaaacatcaaaccaagccctcgtggagagcttgaaatcactgatgttaacaaggcttacttagatcgtggagatctttctgttgagttgatggggcgtggctttgcttggttggatacaggcactcacgaaagcctcctgcaagctgctcaatacattgaaacggttcaacgtttgcaaaatgtccaagtggctaaccttgaggaaattgcctatcgtatgggctacataactaaagaaaaggtcctcgaattggcacaaccgtttaagaagaatgaatacggacaatatttgctccgtttgattggagatgcataa

SEQ ID NO: 7 - protein, 382 aa, Escherichia coli
MSLKEKTQSLFANAFGYPATHTIQAPGRVNLIGEHTDYNDGFVLPCAIDYQTVISCAPRDDRKVRVMAADYENQLDEFSLDAPIVAHENYQWANYVRGVVKHLQLRNNSFGGVDMVISGNVPQGAGLSSSASLEVAVGTVLQQLYHLPLDGAQIALNGQEAENQFVGCNCGIMDQLISALGKKDHALLIDCRSLGTKAVSMPKGVAVVIINSNFKRTLVGSEYNTRREQCETGARFFQQPALRDVTIEEFNAVAHELDPIVAKRVRHILTENARTVEAASALEQGDLKRMGELMAESHASMRDDFEITVPQIDTLVEIVKAVIGEKGGVRMTGGGFGGCIVALIPEELVPAVQQAVAEQYEAKTGIKETFYVCKPSQGAGQC

SEQ ID NO: 8 - protein, 388 aa, Streptococcus thermophilus
MNTSQLREKFKEVFGVEADHTFFSPGRINLIGEHTDYNGGNVLPVAITLGTYGAARKRDDKVLRFFSANFEEKGIIEVPLENLRFEKEHNWTNYPKGVLHFLQEAGHTIDSGMDIYIYGNIPNGSGLSSSSSLELLIGVIVEKLYDIKLERLDLVKIGKQTENDFIGVNSGIMDQFAIGMGADQCAIYLDTNTLKYDLVPLDLKDNVVVIMNTNKRRELADSKYNERRAECETAVSELQEKLDIQTLGELDFLTFDAYSYLIKDENRIKRARHVVLENQRTLQARKALEAGDLEGFGRLMNASHVSLEYDYEVTGLELDTLAHTAWEQEGVLGARMTGAGFGGCAIALVNKDKVEDFKKAVGQRYEEVVGYAPSFYIAEVTGGSRVLD

SEQ ID NO: 9 - protein, 347 aa, Thermus thermophilus
MGFQEVYGVLPQASAQAPGRVNLLGEHTDYQEGYVLPTPIPYFTRVEAAPLEGAVEAFSENLGELRARPLSSPPQGDFLDYLLGVVRALREAGHEVAGARFYVRSDLPMGAGLSSSAALEVAALRALRTLYRLPLSDLEVARLAQKAEVEYVGVRCGIMDQMAASLGQPGQALFLDTRTLAYENLPLPPGVRVAVLDLGLGRRLAEAGYNRRRQEAEEAAKRLGVRSLRDVADLCLVESLPSPLDRRARHVVSENLRVLRGVEALRRGDARAFGELMTQSHRSLAQDYEVNLPELDALVEEALRAGAYGAKLTGAGFGGAVVALVAESRFPAFREALARRFPDLKVL

SEQ ID NO: 10 - protein, 352 aa, Pyrococcus furiosus
MSKITVKSPGRVNLIGEHTDYTYGYVMPMAIDLYTIITAEKYDKVQLYSEHFNEEKTFTLDNLTKEGSWIDYVKGVLWVLIQEGYKIGGLKGKITGDLPLGAGLSSSASFEVGILEVLNQLYNLNIDPLKKALLAKKAENEFVGVPCGILDQFAVVFGKKDNVIFLDTQTLQYEYIPFPKDVSVLVFYTGVKRELASSEYAERKRIAEESLRILGKESSKEVTEKDLGKLPPLHRKFFSYIVRENARVLEVRDALKEGDIEKVGKILTTAHWDLAENYRVSCEELDFFVKKAMELGAYGARLTGAGFGGSAIALVDKDKAKTIGDAILREYLAKFSWKAKYFVVKPSDGVGV

SEQ ID NO: 11 - protein, 390 aa, Artificial Sequence
  Synthetic
XXXXXXXXXXFXEVFGVLAXHTIQAPGRVNLIGEHTDYNDGYVLPMAIDYFTIISAAPRDDKXLRXXVAXFSENLXEEKXXXLIXXXKEGNWINYVKGVLKXLQEAGHSIGXGLDIYISGNLPNGAGLSSSASLEVAILXVLNQLYXLPLDXLDIALLAQKAENEFVGVNCGIMDQFASALGKKDNAIFLDTRTLAYEXVPLPXKGVAVVIINTNLKRELADSEYNERRXECETAARXLQXKSXLRDVTELDLLXXXXXXXXXLPPILXKRARHIVSENARVLEARDALEXGDLEKFGELMTXSHXSLADDYEVTLPELDTLVEXALELGGXAYGARLTGAGFGGCAIALVXKDKVPAFKDAVARRYEAKXGWKXSFYVXKPSXGAGVXX

SEQ ID NO: 12 - protein, 386 aa, Artificial Sequence
  Synthetic
XXXXXXXXXXFXEVFGVLAXXTXXSPGRVNLIGEHTDYNXGYVLPMAIXLFTXIXAXKXDXKVXXFSEXXXEEKXXXXXXXXLXXXXKEGXWIDYLKGVLXXLXEAGHXIAXGLKXYIXGDLPLGAGLSSSASLEVAILXILXXLYXLXLDXLDLAKLAKKAENEFVGVXCGIMDQFAIXLGXXXNAIFLDTNTLXYEXIPLPXXVXVLVIXXXGLKRELADSXYNERKXXAEEAXXKKLGISXLKEVXDLXLXXXXXXXXXXLPXXXXKRARHVVXENXRVLXARDALKXGDIEAFGKLMTXSHXSLAXDYEVSXXELDXLVXXALEXGAYGARLTGAGFGGAAIALVKDKXXXXFKDAIARRYXDLXXWXXXFFIXXXSXGXXV

SEQ ID NO: 13 - protein, 382 aa, Escherichia coli
  MISC_FEATURE (370)..(370): X can be P, M, Y, or F
MSLKEKTQSLFANAFGYPATHTIQAPGRVNLIGEHTDYNDGFVLPCAIDYQTVISCAPRDDRKVRVMAADYDNQLDEFSLDAPIVAHENYQWANYVRGVVKHLQLRNNSFGGVDMVISGSVPQGAGLSSSASLEVAVGTVLQQLYHLPLDGAQIALNGQEAENQFVGCNCGIMDQLISALGKKDHALLIDCRSLGTKAISMPKGVAVVIINSNFKRTLVGSEYNTRREQCETGARFFQQPALRDVTIEEFNAVAHELDPIVAKRVRHILTENARTVEAASALEQGDLKRMGELMAESHASMRDDFEITVPQIDTLVEIVKAVIGEKGGVRMTGGGFGGCIVALIPEELVPAVQQAVAEQYEAKTGIKETXXVCKPSQGAGQC

SEQ ID NO: 14 - protein, 388 aa, Streptococcus thermophilus
  MISC_FEATURE (346)..(346): X is T or L
MNTSQLREKFKEVFGVEADHTFFSPGRINLIGEHTDYNGGNVLPVAITLGTYGAARKRDDKVLRFFSANFDEKGIIEVPLENLRFEKEHNWTNYPKGVLHFLQEAGHTIDSGMDIYIYGSIPNGSGLSSSSSLELLIGVIVEKLYDIKLERLDLVKIGKQTENDFIGVNSGIMDQFAIGMGAEQCAIYLDSNTLKYDLIPLDLKDNVVVIMNTNKRRELAGSKYNERRAECETAVSELQEKLDIQTLGELDFLTFDAYSYLIKDENRIKRARHVVLENQRTLQARKALEAGDLEGFGRLMNASHVSLEYDYEVTGLELDTLAHTAWEQEGVLGARMTGAGFGGCAXALVNKDKVEDFKKAVGQRYEEVVGYAPSXXIAEVSGGSRVLD

SEQ ID NO: 15 - protein, 347 aa, Thermus thermophilus
MGFQEVYGVLPQASAQAPGRVNLLGEHTDYQEGYVLPTPIPYFTRVEAAPLEGAVEAFSENLGELRARPLSSPPQGDFLDYLLGVVRALREAGHEVAGARFYVRSDLPMGAGLSSSAALEVAALRALRTLYRLPLSDLEVARLAQKAEVEYVGVRCGIMDQMAASLGQPGQALFLDSRTLAYENLPLPPGVRVAVLDLGLGRRLAEAGYNRRRQEAEEAAKRLGVRSLRDVADLCLVESLPSPLDRRARHVVSENLRVLRGVEALRRGDARAFGELMTQSHRSLAQDYEVNLPELDALVEEALRAGAYGAKLTGAGFGGAVVALVAESRFPAFREALARRFPDLKVL

SEQ ID NO: 16 - protein, 352 aa, Pyrococcus furiosus
  MISC_FEATURE (312)..(312): X is T or L
MSKITVKSPGRVNLIGEHTDYTYGYVMPMAIDLYTIITAEKYDKVQLYSEHFNEEKTFTLDNLTKEGSWIDYVKGVLWVLIQEGYKIGGLKGKITGDLPLGAGLSSSASFEVGILEVLHQLYNLNIDPLNKALLAKKAENEFVGVPCGILDQFAVVFGKKDNVIFLDSQTLQYEYIPFPKDVSVLVFYTGVKRELASSEYAERKRIAEESLRILGKESSKEVTEKDLGKLPPLHRKFYGYIVRENARVLEVRDALKEGDIEKVGKILTTAHWDLAENYRVSCEELDFFVKKAMELGAYGARLTGAGFGGSAXALVDKDKAKTIGDAILREYHAKFSWKAKXXVVKPSDGVGV

SEQ ID NO: 17 - protein, 389 aa, Artificial Sequence
  Synthetic
XXXXXXXXXXFXEVFGVLAXHTIQAPGRVNLIGEHTDYNDGYVLPMAIDYFTIISAAPRDDKXLRXXVAXFSENLXEEKXXXLIXXXKEGNWINYVKGVLKXLQEAGHSIGXGLDIYISGSLPNGAGLSSSASLEVAILXVLHQLYXLPLDXLDIALLAQKAENEFVGVNCGIMDQFASALGKKDNAIFLDSRTLAYEXIPLPXKGVAVVIINTNLKRELAGSEYNERRXECETAARXLQXKSXLRDVTELDLLXXXXXXXXXLPPILXKRARHIVSENARVLEARDALEXGDLEKFGELTXSHXSLADDYEVTLPELDTLVEXALELGGXAYGARLTGAGFGGCAXALVXKDKVPAFKDAVARRYEAKXGWKXSXXVXKPSXGAGVXX

SEQ ID NO: 18 - protein, 386 aa, Artificial Sequence
  Synthetic
XXXXXXXXXXFXEVFGVLAXTXXSPGRVNLIGEHTDYNXGYVLPMAIXXLFTXIXAXKXDXKVXXFSEXXXEEKXXXXXXXXLXXXXKEGXWIDYLKGVLXXLXEAGHXIAXGLKXYIXGDLPLGAGLSSSASLEVAILXILXXLYXLXLDXLDLAKLAKKAENEFVGVXCGIMDQFAIXLGXXXNAIFLDTNTLXYEXIPLPXXVXVLVIXXXGLKRELAGSXYNERKXXAEEAXXKKLGISXLKEVXDLXLXXXXXXXXXXLPXXXXKRARHVVXENXRVLXARDALKXGDIEAFGKLMTXSHXSLAXDYEVSXXELDXLVXXALEXGAYGARLTGAGFGGAAXALVKDKXXXXFKDAIARRYXDLXXWXXXXXIXXXSXGXXV

SEQ ID NO: 19 - protein, 420 aa, Pyrococcus furiosus
MKAIILAAGKGERLRPLTDDRPKVVLKIANKPIISYVLENLDPFVDEFIIVVKYMKEKVIDLLGDEFRGKPITYVEQGEEEGTAAAVYSVKEFIESNEEFFVVNGDLYFEPDAVKGLLHVFKKEKGDAGIVVKEFENLSQYGMVEVENGKVKGIIEKPGNVKGYANLGIYIFKSDVFDYIENTEISERGEYEITDTINLMIKDGKSVVYYNYEGFWSDIGRPWDLLEVNEYILKTKLKHEIRGVVEEGATIIPPVEIGEGTIVRAGSYIIGPVKIGKNCRIGPNCYIRPYTSIGDNCHIGNAVEVKNSIIMDNSNAPHLNYVGDSIIGENTNLGAGTITANLRHDKGTIKVEVKGKLEDSGRRKLGAIIGHNVKVGINVTIYPGRKIGSNSFIGPGVIVDKNIPQNVLVTVKQEKKVIEK

SEQ ID NO: 20 - protein, 348 aa, Thermus thermophilus
MKGLILAAGRGTRLRPLTHTRPKPAIRVAGRPIIHYAVENLLEAGVREIGVVVSPETERDLKVALEGYPVRYVLQEEPQGLAHAVDVARDFLGESPFVLYLGDNLFQKGIRRFLEAFKPGVSAVIALVRVEDPRQFGVAVLEGNRVVRLLEKPKDPPSDLAVAGVYVFSPEVLEVVRGLKPSARGEYEITDAIQGLIDRGKTVVGVEVEGWWKDTGRHQDLLDANRLLLEELTPKVEGDVVESQLTGRVVVEKGAKVVRSTVIGPAHIGAGAVVEEAFVGPFTSVGPRARVVRSEVEYSLLEEEAVVEEVRLRLQECILGVRAEVKSRDGLPRAHRLILGDLSQVELA

SEQ ID NO: 21 - protein, 289 aa, Streptococcus thermophilus
MKGIILAGGSGTRLYPLTRAASKQLMPIYDKPMIYYPLSTLMLAGIKEILIISTSQDLPRFEELLGDGSEFGISLSYAVQPSPDGLAQAFIIGEDFIGDDSVALVLGDNIFHGNGLSAMLQRAEAKEKGATVFGYQVKDPERFGVVEFDDDMNAISIEEKPEHPKSNFAVTGLYFYDNDVVEIAKNIKPSPRGELEITDVNKAYLDRGDLSVELMGRGFAWLDTGTHESLLQAAQYIETVQRLQNVQVANLEEIAYRMGYITKEKVLELAQPFKKNEYGQYLLRLIGDA

SEQ ID NO: 22 - protein, 427 aa, Artificial Sequence
  Synthetic
MKGIILAAGKGTRLRPLTXXRPKXLIKIAXKPIIHYCLENLLDAGVKEIIIVVSXXXEKXIDLLLDDXXXXGXPISYVLQXEPDGLAXAVXIAKDFIGDXXXXLVLGDNLFXXXXXIKXLLXAFKKEKXXAXIXLXXVEDPXQFGVVELEXXKXVIXIIEKPXXPXSXFAVXGIYIFXXDVLEIIKNIKPSXRGEYEITDXINALIDRGKSVVXLXXEGXFWXDTGRHXDLLDANXYILXXXXXXXIXXVVEEXITXRMXVEXGEKVIXXAXXFXXXXXXXXXXXXXXXXXXXXXXXXXXXXXXXXGNAVLXXXXIAXXXSXAPXXXXVXXXIXXXXXXXXAXXXXXXLRXXXXIXVXXXXKXXDXXXRXXXXIIGXXXXVXIXXXXXXXXXXXXXXXXXXXXXXXXXXXXXXXXXXXXXXXXXXXX

(19) United States
(12) Patent Application Publication
(10) Pub. No.: US 2016/0272953 A1
(43) Pub. Date: Sep. 22, 2016

(54) Activated Sugars

(71) Applicant: ZuChem, Inc., Chicago, IL (US)

(72) Inventors: Ryan Woodyer, Normal, IL (US); Paul Taylor, Arlington Heights, IL (US); David Demirjian, Hinsdale, IL (US)

(21) Appl. No.: 15/172,836

(22) Filed: Jun. 3, 2016

Related U.S. Application Data
(63) Continuation of application No. 13/817,888, filed on Oct. 7, 2013, now abandoned.
(60) Provisional application No. 61/375,488, filed on Aug. 20, 2010.

(51) Int. Cl. C12N 9/12; C12P 19/02; C12P 19/18

(52) U.S. Cl. CPC C12N 9/1241; C12P 19/18; C12P 19/02; C12Y 207/07

(57) ABSTRACT

Kinase and nucleotidyltransferase enzymes for the production of activated sugars have been developed. These enzymes have improved stability for industrial application and relaxed specificity towards a variety of sugars. These enzymes are useful in, for example, the production of diverse NDP-sugars for glycosylation of aglycones of interest, production of oligosaccharides, production of other important glycoylated sugars, and in drug discovery applications.

### PRIORITY

This application is a continuation of U.S. Ser. No. 13/817,888, filed on Oct. 7, 2013, which is a 371 application of PCT/US11/48642, filed on Aug. 22, 2011, which claims the benefit of U.S. Ser. No. 61/375,488, filed on Aug. 20, 2010, all of which are incorporated by reference in their entirety.

### GOVERNMENT INTERESTS

This invention was made with government support under N.I.H. Grant 2R44-GM079004. The U.S. Government has certain rights in this invention.

### BACKGROUND OF INVENTION

### The Importance of Sugar Ligands

Natural product glycosylation is becoming increasingly important in the discovery of new pharmaceutical compounds and the development of important new food ingredient and other industrial chemicals. Many biologically active natural products owe their bioactivity at least in part to glycosylation and many are naturally glycosylated secondary metabolites. The sugar attachments impart a variety of important activities. [1-5] For example, sugar moieties can be critical to the inhibition of key functions such as DNA processing (e.g., antracyclines like daunorubicin and aclarubicin), translation (e.g., erythromycin) and cell wall synthesis (e.g., vancomycin). They can be involved in membrane recognition (e.g., amphotericin and novobincin) and DNA recognition (e.g., calicheamicin). They can also be important in the formation of protein complexes (e.g., cardiac glycosides such as digitoxin). It has been postulated that there is a large opportunity to discover many new drugs through the use of glycosylation by both altering glycosylation patterns on natural products and attaching sugar ligands to drug candidates that are not normally glycosylated. In food applications, sugars are main components of sweeteners. Different sugar constituents with different sweetness profiles of high intensity sweeteners such as Luo Han Guo (Monk Fruit) and Stevia have different sugars attached to their core structures (REF). Oligosaccharides such as globotriose and others have a variety of important nutritional and health properties. Finally, different sugars attached to polypeptides and proteins can have an important effect on the activity and distribution of the molecules (REF).

### Methods to Modify Natural Product Glycosylation

Although there is a tremendous desire to explore glycosylation, general methods for creating the diversity of glycosylation have been extremely difficult to develop—to a large extent because the required building blocks and activated sugar intermediates needed to carry out this research cannot currently be made. Only a limited number of highly specific methods have been explored[6-10]:

1. Total Synthesis or Semi-Synthesis. Traditionally, chemists have used total synthesis of analogs or synthetic modification of intermediates usually produced via fermentation as a tool for exploring glycochemical modifications. Total or semi-synthetic methods have been extremely limited due to the enormous structural complexity of many glycosylated natural products and the corresponding difficulties associated with their regio- and stereo-specific chemical glycosylation. As a result, often only a limited number of products can be made and only one product at a time can be explored because of their complexity. Thus, medicinal chemists have often avoided or ignored studying modified glycosylation in their drug discovery efforts.

2. Pathway Engineering and Bioconversion Another method that has been explored is to modify existing biological pathways to generate different but related glycochemical products. For example, in vivo methods to alter glycosylation of macrolides and other molecules[11-14] have been explored using pathway engineering (or ‘combinatorial biosynthesis’)[11-14] and bioconversion [15, 16] Disruption of genes leading to the biosynthesis of dTDP-D-desosamine, a precursor to pikromycin, methymycin, and related macrolides in S. venezuluae, led to macrolides with new sugar moieties attached. In addition, introduction of biosynthetic genes from other pathways (Δdesl, calS13—which incorporates a sugar 4-aminotransferase from M. echinospora) led to further diversity in glycosylation. Bioconversion has also been applied for the generation of novel avermectin derivatives.[17] In this example, combinations of TDP-D-desosamine (pikromycin/methymycin, S. venezuluae) and TDP-L-oleandrose (avermectin, S. avermitilis) biosynthetic genes were assembled in a non-producing host S. lividans engineered to express the avermectin glycosyltransferase gene, avrB. Upon feeding this host the avermectin aglycon, novel D-sugar substituted avermectins were produced. These examples highlight the promiscuity displayed by glycosyltransferases of secondary metabolism but at the same time are limited in their breadth of application. [4, 5]

While these methods are potentially useful in specific instances there are at least two major hurdles to using them in a broad fashion. First, the utility of the systems are limited to enzymes that express well and are active in the systems that are used. Second, the systems are limited by the ability of the cells to transport the substrates and products into and out of the cell.

3. Natural Enzymatic System for Carbohydrate Attachment. The biological method for carbohydrate attachment for many natural products generally involves three steps. First is activation at the 1-position using a sugar kinase (such as GalK) to phosphorylate the carbohydrate. This step is followed by a nucleotidyltransferase (such as EP) that forms an activated NDP-sugar. Then, these activated carbohydrates coupled to an aglycone (or another sugar) through the use of a glycosyltransferase (GlyT). By harnessing this method one could take advantage of the combined flexibility of chemical synthesis of unique sugar precursors with natural or engineered substrate promiscuity of enzymes to make an activated sugar library (using sugar kinases, and nucleotidyltransferases) and attach them to various natural product aglycones with naturally promiscuous glycosyltransferases (“GlyT”) as shown in Figure A1. In this approach, natural and “unnatural” sugar precursors could be chemically (or enzymatically) synthesized and attached to various aglycons with the natural biological three enzyme system.

It could even allow for the efficient incorporation of sugars with ‘reactive handles’ (e.g. azides, thiols, ketones, aminooxy substituents) that can later be modified, to further expand the diversity of a chemical library. This method would also allow for the simple scale-up of these chemicals that would otherwise be difficult to achieve. If the right enzyme could be discovered or developed it should be potentially possible to utilize this as either in vivo or in vitro as either a sequential series of enzymatic reactions or as a combined one- or two-pot synthesis.

It is this third method that provides the most potential for both the drug discovery chemist wanting to generate large libraries of glycosylated aglycones of interest and the simplified scaled production of these compounds. Unfortunately, although there has been some work to explore, a number of factors have prevented the practical use of this technology to generate broad libraries of glycosylated compounds. One factor has been the lack broad substrate specificity sugar-1-kinases and the stability of the enzymes that can be used with a variety of sugar moieties. Of special note is the lack of a system exists for attachment of L-sugar and azido-sugar moieties. L-Sugars are present in many bioactive natural products, are not readily metabolized, and can result in lower toxicity, making them medically relevant. A second important hurdle is the availability of a stable enzyme system that can be used in a practical industrial environment to produce the large quantities of product needed for commercial application.

### BRIEF DESCRIPTION OF THE FIGURES

Figure A1 shows enzymatic glycosylation of molecules using activated sugars.

Figure B shows analysis of GalKMLYH.

FIG. 1-1 shows a DNS reaction with positive controls circled.

FIG. 1-2 shows TLC analysis of sugar-1-kinase reaction products.

FIG. 2-1 shows high throughput TLC screen for nucleotidyltransferase activity.

FIG. 2-2 shows a malachite green assay for nucleotidyltransferase activity.

FIG. 3-1 shows a DNS assay of thermostable kinases.

FIG. 3-2 shows sugar-1-kinase conversion at various temperatures.

FIG. 3-3 shows sugar-1-kinase conversion of alternative substrates.

FIG. 4-1 shows sugar-1-kinase mutant conversion.

FIG. 4-2 shows sugar-1-kinase mutants.

FIG. 4-3A-B show sugar-1-kinase activity assays.

FIG. 4-4 shows sugar-1-kinase-PK27 enzyme purification.

FIG. 4-5A-B shows testing for broad sugar-1-kinase-PK27 substrate specificity.

FIG. 4-6 shows production of L-glucose-1-phosphate.

FIG. 5-1 shows a SDS-PAGE analysis of purified nucleotidyltransferases (NT).

FIG. 5-2 shows confirmation of nucleotidyltransferase activity with dTTP and Gal-1-P by TLC and malachite green assay.

FIG. 6-1 shows a coupled kinase and nucleotidyltransferase reaction.

FIG. 6-2 shows a malachite green assay for analysis of nucleotidyltransferase activity at different temperatures.

FIG. 6-3 shows a TLC analysis of coupled reaction.

FIG. 7-1 shows a homology comparison of wild-type sugar-1-kinases from S. thermophilus (St), Thermus thermophilus (Tt) and Pyrococcus furiosus (Pf) with E. coli Galactose-1-phosphate.

FIG. 7-2 shows a homology comparison of mutant sugar-1-kinases from S. thermophilus (St), Thermus thermophilus (Tt) and Pyrococcus furiosus (Pf) with E. coli Galactose-1-phosphate.

FIG. 7-3 shows a homology comparison of nucleotidyl transferases from Pyrococcus furiosus, T. thermophilus, and S. thermophilus.

FIG. 8-1 shows SEQ ID NOs:4, 5, 6, 19, 20, and 21.

FIG. 8-2 shows SEQ ID NOs:1, 2, 3, 8, 9, and 10.

### SUMMARY OF THE INVENTION

One embodiment of the invention provides an isolated sugar-1-kinase, wherein the isolated sugar-1-kinase has sugar-1-kinase activity in a sugar-1-kinase assay and has a T₅₀ half-life at 30° C. of greater than 10 minutes. The sugar-1-kinase assay can be a 3,5-dinitrosalicylic acid (DNS) assay, a thin layer chromatography assay or a high-performance liquid chromatography assay. The isolated sugar-1-kinase can comprise at least 90% amino acid sequence identity to SEQ ID NO:12, SEQ ID NO:8, SEQ ID NO:9, or SEQ ID NO:10, wherein the isolated sugar-1-kinase has sugar-1-kinase activity in a 3,5-dinitrosalicylic acid (DNS) assay. The isolated sugar-1-kinase can comprise:
(a) SEQ ID NO:8 with the following mutations:
   (i) N120S; D183E; T191S; Y376F; and T381S;
   (ii) E71D and VI99I;
   (iii) D221G; or
   (iv) a combination of one or more of the following mutations: N120S; D183E; T191S; Y376F; T381S; E71D; VI99I; D221G; I341T; I341L, F375P F375M; F375Y; Y376K; Y376T; Y376P; and Y376F;
(b) SEQ ID NO:10 with the following mutations:
   (i) N119H; K130N; S239G; F238Y; and I312L;
   (ii) I312T and L332H;
   (iii) Y341P and F342K;
   (iv) Y341M and F342T;
   (v) I312T; L332H; Y341P; and F342K; or
   (vi) a combination of one or more of the following mutations: N119H; K130N; S239G; F238Y; I312L; I312T; L332H; Y341P; F342K; and Y341M; F342T; T168S; Y341P; Y341M; Y341F; F342K; F342T; F342P; F342Y;
(c) SEQ ID NO:9 with the following mutation: T177S; or
(d) SEQ ID NO:12 with a combination of one or more of the following mutations: D222G; I348T; I348L; F377P; F377M; F377Y; F378K; F378T; F378P; or F378Y.
   The sugar-1-kinase can comprise at least 90% amino acid sequence identity to SEQ ID NO:14, SEQ ID NO:15, SEQ ID NO:16; or SEQ ID NO:18, wherein the isolated sugar-1-kinase has sugar-1-kinase activity in, for example, a 3,5-dinitrosalicylic acid (DNS) assay, a TLC assay or a HPLC assay.

Another embodiment of the invention provides a polynucleotide that encodes a sugar-1-kinase of the invention.

Yet another embodiment of the invention is an expression vector or host cell that comprises a sugar-1-kinase polynucleotide of the invention.

Still another embodiment of the invention provides an isolated nucleotidyltransferase comprising at least 90% amino acid sequence identity to SEQ ID NO:19, SEQ ID NO:20, SEQ ID NO:21, or SEQ ID NO:22, wherein the isolated nucleotidyltransferase has nucleotidyltransferase activity in a inorganic phosphate assay. The isolated nucleotidyltransferase can have a T₅₀ half-life at 30° C. of greater than 10 minutes.

Another embodiment of the invention provides a polynucleotide encoding the a nucleotidyltransferase of the invention.

Yet another embodiment of the invention provides an expression vector or host cell that comprises a nucleotidyltransferase polynucleotide of the invention.

Still another embodiment of the invention provides a method of phosphorylating one or more sugars. The method comprises contacting the sugars with a sugar-1-kinase of the invention, wherein phosphorylated sugar-1-phosphates are produced. The reaction temperature can be greater than 30° C. and the conversion rate of sugar to sugar-1-phosphate can be greater than 50%. The sugar can be an L-sugar or a D-sugar. The sugar can be D-galactose, L-galactose, L-glucose, D-glucose, D-glucoronate, L-rhamnose, D-arabinose, L-arabinose, L-xylose, D-xylose, L-ribose, D-ribose, D-fucose, D-fucose, L-fucose, L-xylose, L-lxyose, D-xylose, L-mannose, D-mannose, L-gulose, 6-azido-D-galactose, or a combination thereof. The sugar-1-phosphates can further be contacted with a nucleotidyltransferase to produce nucleoside-diphosphate (NDP) sugars. The nucleotidyltransferase and the sugar-1-kinase can be contacted with the sugars at the same time or sequentially.

Even another embodiment of the invention provides a method of converting one or more sugar-1-phosphates to nucleoside-diphosphate (NDP) sugars. The method comprises contacting the sugar-1-phosphates with a nucleotidyltransferases of the invention, wherein NDP sugars are produced. The reaction temperature can be greater than 30° C. and the conversion rate of sugar-1-phosphates to NDP sugars can be greater than 50%. The sugar-1-phosphate can be an L-sugar-1-phosphate or a D-sugar-1-phosphate. The sugar-1-phospate can be D-galactose-1-phosphate, L-galactose-1-phosphate, L-glucose-1-phosphate, D-glucose-1-phosphate, D-glucoronate-1-phosphate, L-rhamnose-1-phosphate, D-arabinose-1-phosphate, L-arabinose-1-phosphate, L-xylose-1-phosphate, D-xylose-1-phosphate, L-ribose-1-phosphate, D-ribose-1-phosphate, D-fucose-1-phosphate, D-fucose-1-phosphate, L-fucose-1-phosphate, L-xylose1-phosphate, L-Ixyose-1-phosphate, D-xylose-1-phosphate, L-mannose-1-phosphate, D-mannose-1-phosphate, L-gulose-1-phosphate, 6-azido-D-galactose-1-phosphate, or a combination thereof.

We have successfully developed a platform technology to make activated sugars. Included in this technology are kinases that are capable of attaching a phosphate group to a broad range of sugars as well as nucleotidyltransferases that are capable of taking a nucleotide triphosphate and attaching it to a phosphorylated sugar, thereby creating an activated sugar. These enzymes are stable making them useful for the production of activated sugars. They have been cloned from all the major classes of thermophilic organisms including moderate thermophiles, extreme thermophiles, and hyperthermophiles. Stable enzymes can alternatively be created by using a directed evolution or mutagenesis program. The enzymes are useful to produce sugar-1-phosphates, activated sugars, activated sugar libraries, glycosylated molecules and oligosaccharides. They are also unique in their ability to not only to produce a wide variety of sugar-q-phosphates and activated sugars, but those that incorporate I-sugars and azo-sugars.

### DETAILED DESCRIPTION OF THE INVENTION

### Enzymes Involved in Making Activated Sugars

There are two main enzymes involved in the production of an activated sugar: a sugar kinase and a nucleotidyltransferase (also known as a nucleotidylyl transferase).

1. Kinase. Sugar kinases catalyze the formation of a sugar-1-phosphate from a sugar and ATP. In particular, galactokinases (GalK) have been studied that catalyze the formation of alpha-D-galactose-1-phosphate (Gal-1-P) from D-galactose and ATP. Yet, the kinases characterized to date are known to be specific for one or only a few monosaccharides.[18-20] Moreover, in all C-1 kinases studied previously, a strict adherence to either D-sugars (GaIK and glycogen phosphorylases),[18-21] or L-sugars (as in fucokinase)[22] was observed.

In order to use any of these kinases to generate a randomized sugar phosphate library, their monosaccharide substrate promiscuity must be enhanced. Prior work by Thorson and coworkers demonstrated that a mutagenesis approach could be useful in broadening substrate activity of the E. coli GalK enzyme In these experiments one particular GalK mutant (Y371 H)[23-25] was identified that displayed modified kinase activity toward additional sugars including D-talose, D-galacturonic acid, L-altrose, and L-glucose (the only tested L-sugar seen to be used), all of which failed as wild-type GalK substrates.[20, 24-27] In addition, the GalK Y371H mutant had enhanced turnover with the natural substrates of the wild-type enzyme. Thorson and coworkers then modeled glucose into the E. coli GalK active site (using the L. lactis structure as a template) which led to the design of a GalK M173L mutant capable of efficient dual gluco- and galacto- kinase turnover. Using these methods, a single GalK variant carrying both the M173L and Y371H mutations (GalKMLYH) was constructed.

Testing was carried out using the only previously identified enzyme capable of phosphorylating a broad range of sugars—the engineered E. coli GalKMLYH [48]. This mutant enzyme has a broadened substrate range and has previously reported to be capable of converting ˜1 milligram quantities of sugars and derivatives to their corresponding 1-phosphates at various yields, including 25% conversion of L-glucose. [21] However, this low conversion and productivity were only achievable at the low substrate concentrations (1.5 g/L) and high concentrations of purified enzyme (0.6 g/L). The specificity of this E. coli GalK mutant was examined with additional L-sugars and suitability of this enzyme for commercial production. Of importance was the ability to demonstrate that it could be used in an industrial environment.

The GalK mutant was expressed and purified as previously described. [21] but proved to be an extremely unstable enzyme. The GalK enzyme activity was initially tested for 3 hrs at room temperature on a small subset of sugars including D-galactose, 2-deoxy-D-galactose and D-glucose, all of which were previously known substrates. No activity was observed with any of the substrates after the enzyme had been stored at room temp for 3 hr. Subsequently, the enzyme was tested for its stability by incubation at various temperatures followed by assay with 12 mM ATP, 3.5 mM Mg²⁺, and 8 mM D-galactose followed by DNS reducing sugar assay of the remaining D-galactose. It became immediately clear that the engineered enzyme only maintained activity for more than a few hours if kept at 16° C. or cooler and lost all activity within 1 hr at 30° C.

The GalKMLYH enzyme was finally tested at 16° C. for the conversion of several other L-sugars using partially purified cell extract from the overexpressing E. coli strain and typical reaction conditions. As displayed in Figure B, the GalK mutant did not display significant activity on any of the substrates tested (L-arabinose, L-fucose, L-glucose, L-gulose, L-mannose, L-rhamnose, L-ribose, L-xylose), even after 5 hrs of incubation.

Thus it was determined that it was not suitable to use the E. coli GalKMLYH mutant for commercial production of sugar-1-phosphates or activated sugars, since it was neither stable enough, nor active enough on L-sugars.

While the GalKMLYH and the two individual mutants work to produce small trace quantities of some sugars, their stability proved extremely problematic. It was determined these enzymes were not useful for producing sufficient quantities of material. Additionally, although it had some increased substrate range, the breadth of this range was not sufficient for a general industrial tool.

2. Nucleotidylyltransferase. Nucleotidlylytransferases catalyze the attachment of an NDP group to the phosphorylated sugar, thereby producing an active sugar. As in the case of the kinase, some research has been carried out to expand the substrate specificity of the enzyme. Out of the many available nucleotidyltransferases, structure-based engineering has previously been demonstrated with the rm/A-encoded alpha-D-glucopyranosyl phosphate thymidylyltransferase (Eₚ) from Salmonella enterica LT2.[28] Nucleotidlylytransferase catalyzes the conversion of alpha-D-glucopyranosyl-1-phosphate (Glc-1-P) and dTTP to dTDP-alpha-D-glucose (dTDP-Glc) and pyrophosphate (PPᵢ) via a single sequential displacement mechanism.[29] This enzyme displayed promiscuity toward both its nucleotide triphosphate (dTTP and UTP) and the sugar phosphate substrates.[30-32] Yet sterics, ring formation, and/or electrostatic limitations prohibited the use of nucleotidlylytransferase in a broad fashion.

A structure-based engineering approach led to nucleotidlylytransferase variants capable of utilizing an expanded sugar-1-phosphate set.[29, 33, 34] As with the GalK enzyme, however, this enzyme is also very unstable and difficult to use for the production of anything other than trace amounts of some products.

Thus the main hurdle to getting the kinase and nucleotidyltransferase to work is the lack of stability that they exhibit, making them impractical for use. The development of a stable enzyme is the key step that would ultimately enable the ability to make individual activated sugars, activated sugar libraries for combinatorial chemistry and drug discovery applications, and large quantities of activated sugars for the manufacture of important chemicals, oligosaccharides, intermediates, and pharmaceuticals.

### Gylcosyltransferases

There are a number glycosyltransferases available to generate glycosylated small molecule libraries, protein and peptide glycosides and create oligosaccharides. These glycosyltransferases often have specificity for the acceptor aglycone which is getting glycosylated, but are able to take a variety of activated sugars. One example is the glycosyltransferase GtfE, the first of two tandem glycosyltransferases in vancomycin biosynthesis, which was utilized with 33 natural and ‘unnatural’ NDP-sugars −31 from this set were accepted as substrates (>25% conversion).[35-37]

Given many natural product-associated glycosyltransferases have been shown to be promiscuous (based upon genetic and biochemistry approaches),[3-5] it is anticipated this method will be generally applicable to many natural product scaffolds. This is extremely relevant as the widespread availability of libraries of activated sugars will greatly simplify the synthesis of glycosylated derivatives (using an appropriate glycosyltransferase) from both naturally and synthetically derived aglycons. As the glycosyltransferases are generally promiscuous, it follows that the availability of libraries of NDP-sugars would be of great value to glycochemical research community; not least using these libraries as a tool for the selection of more flexible glycosyltransferases.

Substrate Stereochemistry. Although one might wonder about the promiscuity of GTs towards activated L-sugar substrates, there are many literature examples of GTs accepting NDP-L-Sugars. Several were mentioned above including natural activities for GtfE involved in vancomycin biosynthesis and avrB involved in avermectin biosynthesis.[17] There are many other examples, such as SorF, a GT from the sorangicin biosynthetic gene cluster that showed high flexibility towards UDP- and dTDP-sugars and was able to transfer several sugar moieties including D-glucose, D-galactose, D-xylose, L-rhamnose, and 6-deoxy-4-keto-alpha-D-glucose onto the aglycon.[39] GtfA, B, C, and D as mentioned above are each capable of transferring several different NDP-L-sugars that were tediously synthesized in mg quantity to vancomycin class aglycones.[40] CalG1, a GT responsible for glycosylation of the anticancer enediyne calicheamicin, was capable of transferring a multitude of different TDP-sugars including TDP-L-rhamnose. [41] There are many other examples of GTs using NDP-L-sugars.[42-45] Furthermore, altering the substrate specificity of GTs has proven successful. [46] However, in large part the study of GT substrate specificity with NDP-L-sugars has been limited because the NDP-L-sugars are not available commercially.

All patents, patent applications, and other scientific or technical writings referred to anywhere herein are incorporated by reference herein in their entirety. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

### Polypeptides

As used herein, the singular forms “a,” “an”, and “the” include plural referents unless the context clearly dictates otherwise.

A polypeptide is a polymer of two or more amino acids covalently linked by amide bonds. A polypeptide can be post-translationally modified. A purified polypeptide is a polypeptide preparation that is substantially free of cellular material, other types of polypeptides, chemical precursors, chemicals used in synthesis of the polypeptide, or combinations thereof. A polypeptide preparation that is substantially free of cellular material, culture medium, chemical precursors, chemicals used in synthesis of the polypeptide, etc., has less than about 50%, 40%, 30%, 20%, 10%, 5%, 1% or more of other polypeptides, culture medium, chemical precursors, and/or other chemicals used in synthesis. Therefore, a purified polypeptide is about 50%, 60%, 70%, 80%, 90%, 95%, 99% or more pure. A purified polypeptide does not include unpurified or semi-purified cell extracts or mixtures of polypeptides that are less than 50% pure.

The term “polypeptides” can refer to one or more of one type of polypeptide (a set of polypeptides). “Polypeptides” can also refer to mixtures of two or more different types of polypeptides (a mixture of polypeptides). The terms “polypeptides” or “polypeptide” can each also mean “one or more polypeptides.”

One embodiment of the invention provides one or more of the following sugar-1-kinase polypeptides:
1. Streptococcus thermophilus wild-type sugar-1-kinase (SEQ ID NO:8);
2. Thermus thermophilus wild-type sugar-1-kinase (SEQ ID NO:9);
3. Pyrococcus furiosus wild-type sugar-1-kinase (SEQ ID NO:10);
4. Consensus1 (SEQ ID NO:11), which is a consensus sequence of wild-type E. coli GalK protein (SEQ ID NO:7), SEQ ID NO:8, SEQ ID NO:9, and SEQ ID NO:10.
5. Consensus2 (SEQ ID NO:12), which is a consensus sequence of SEQ ID NO:8, 9, and 10.
6. E. coli mutant GalK protein (SEQ ID NO:13);
7. Streptococcus thermophilus mutant sugar-1-kinase (SEQ ID NO:14);
8. Thermus thermophilus mutant sugar-1-kinase (SEQ ID NO:15);
9. Pyrococcus furiosus mutant sugar-1-kinase (SEQ ID NO:16);
10. Consensus1 (SEQ ID NO:17), which is a consensus sequence of mutant E. coli GalK protein (SEQ ID NO:13), SEQ ID NO:14, SEQ ID NO:15, and SEQ ID NO:16.
11. Consensus2 (SEQ ID NO:18), which is a consensus sequence of SEQ ID NO:14, 15, and 16.

Also included are the following mutant sugar-1-kinase proteins:
1. SEQ ID NO:8 with the following mutations:
   (i) N 120S; D183E; T191S; Y376F; and T381S;
   (ii) E71D and VI99I;
   (iii) D221G; or
   (iv) A combination of one or more of the following mutations: N120S; D183E; T191S; Y376F; T381S; E71D; VI99I; D221G; I341T; I341L; F375P F375M; F375Y; Y376K; Y376T; Y376P; and Y376F.
2. SEQ ID NO:10 with the following mutations:
   (i) N119H; K130N; S239G; F238Y; and I312L;
   (ii) I312T and L332H;
   (iii) Y341P and F342K;
   (iv) Y341M and F342T;
   (v) I312T; L332H; Y341P; and F342K; or
   (vi) A combination of one or more of the following mutations: N119H; K130N; S239G; F238Y; I312L; I312T; L332H; Y341P; F342K; and Y341M; F342T; T168S; Y341P; Y341M; Y341F; F342K; F342T; F342P; F342Y.
3. SEQ ID NO:7 with a combination of one or more of the following mutations: E72D; N120S; VI99I; F370P; F370M; and F370Y.
4. SEQ ID NO:9 with the following mutation: T177S.
5. SEQ ID NO:11 with a combination of one or more of the following mutations: N121S; N143H; T192S; V200I; D222G; I348T; I348L; F377P; F377M; F377Y; Y378K; Y378T; Y378P; Y378F.
6. SEQ ID NO:12 with a combination of one or more of the following mutations: D222G; I348T; I348L; F377P; F377M; F377Y; F378K; F378T; F378P; F378Y.

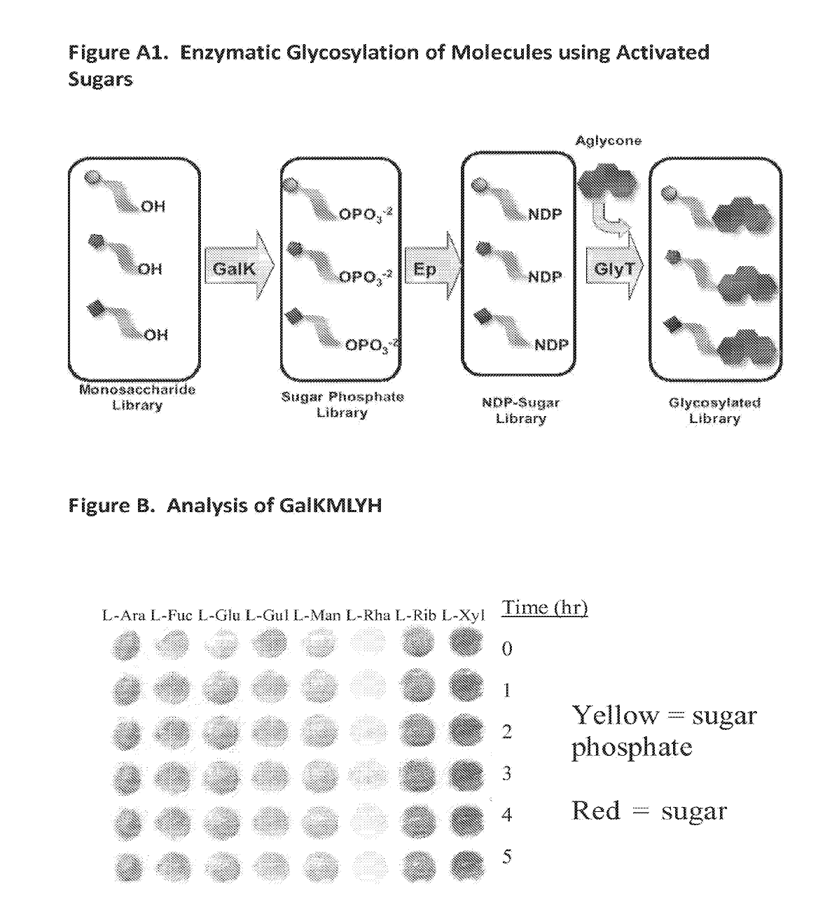

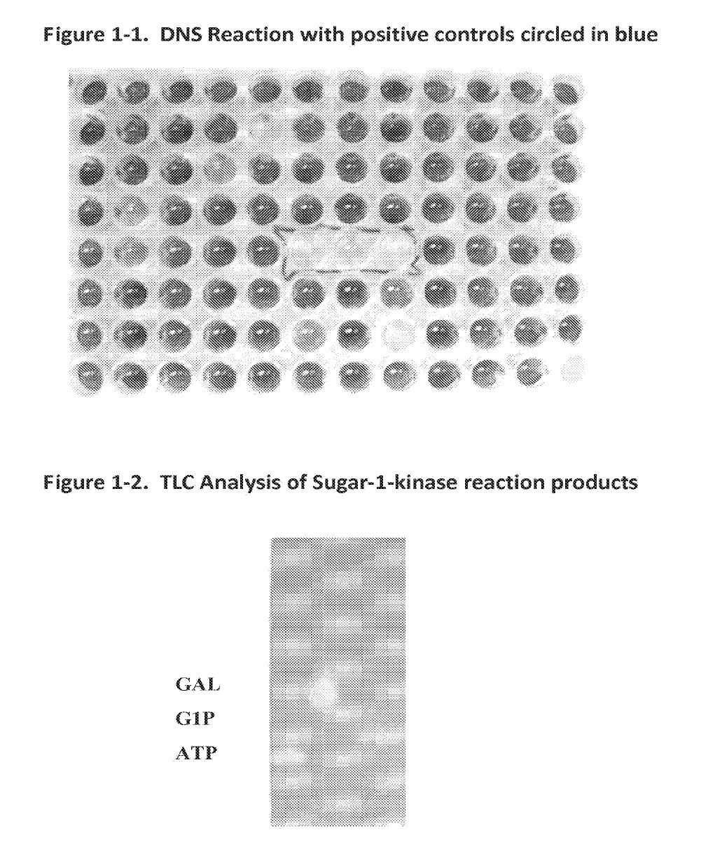

FIGS. 7-1 and 7-2 show the alignment of wild-type (7-1) and mutant (7-2) polypeptides. Consensus1 is the alignment of the SEQ ID NOs:7, 8, 9, and 10. Consensus2 is the alignment of SEQ ID NOs:8, 9, and 10. There are several X's in the consensus sequences. In one embodiment of the invention, an X can stand for any amino acid. In other embodiment of the invention an X can stand for only the amino acids that occur in the corresponding position in SEQ ID NO:7, SEQ ID NO:8, SEQ ID NO:9, and SEQ ID NO:10 (or alternatively only SEQ ID NO:8, SEQ ID NO:9, and SEQ ID NO:11). For example, the X at position 20 of SEQ ID NO:10 and 11 can be K, Q, and D in one embodiment or K, Q, D, and T in another embodiment.

The sugar-1-kinases of the invention can phosphorylate one or more sugars wherein phosphorylated sugar-1-phosphates are produced. 3,5-dinitrosalicylic acid (DNS) assays can be used to detect activity of the sugar-1-kinases. The sugar-1-kinase can be active on any sugar, including for example, D-galactose, L-glucose, L-rhamnose, D-arabinose, L-arabinose, L-xylose, D-xylose, D-fucose, L-fucose, L-mannose, D-mannose, L-gulose, 6-azido-D-galactose, or a combination thereof.

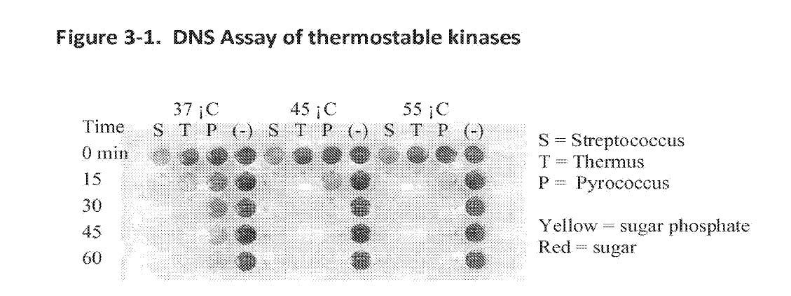

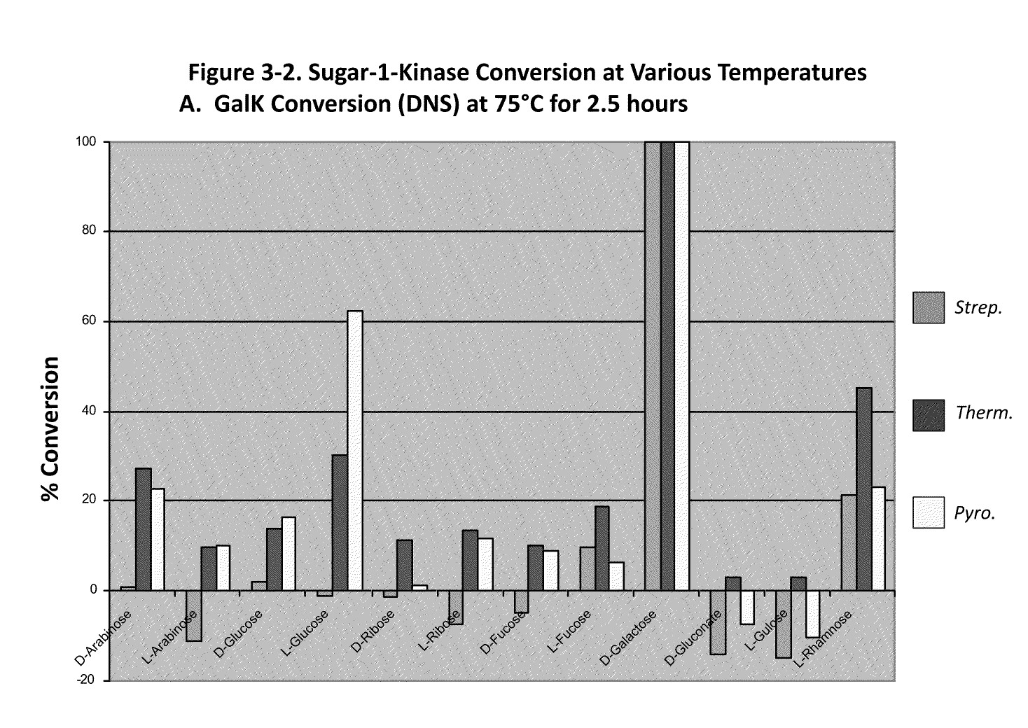

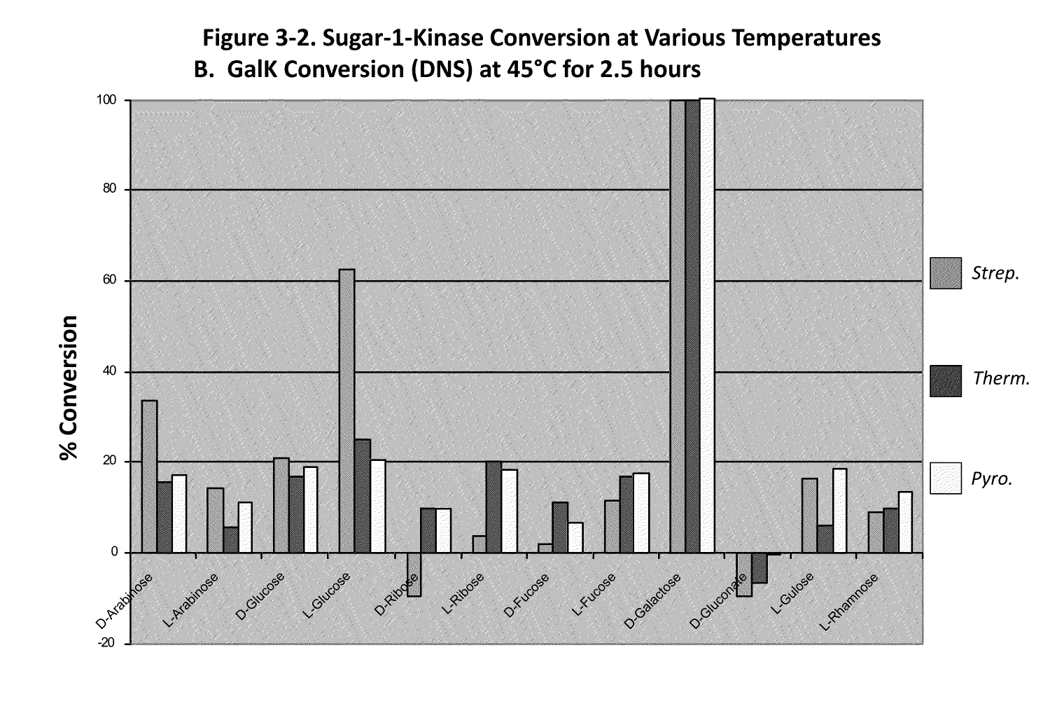

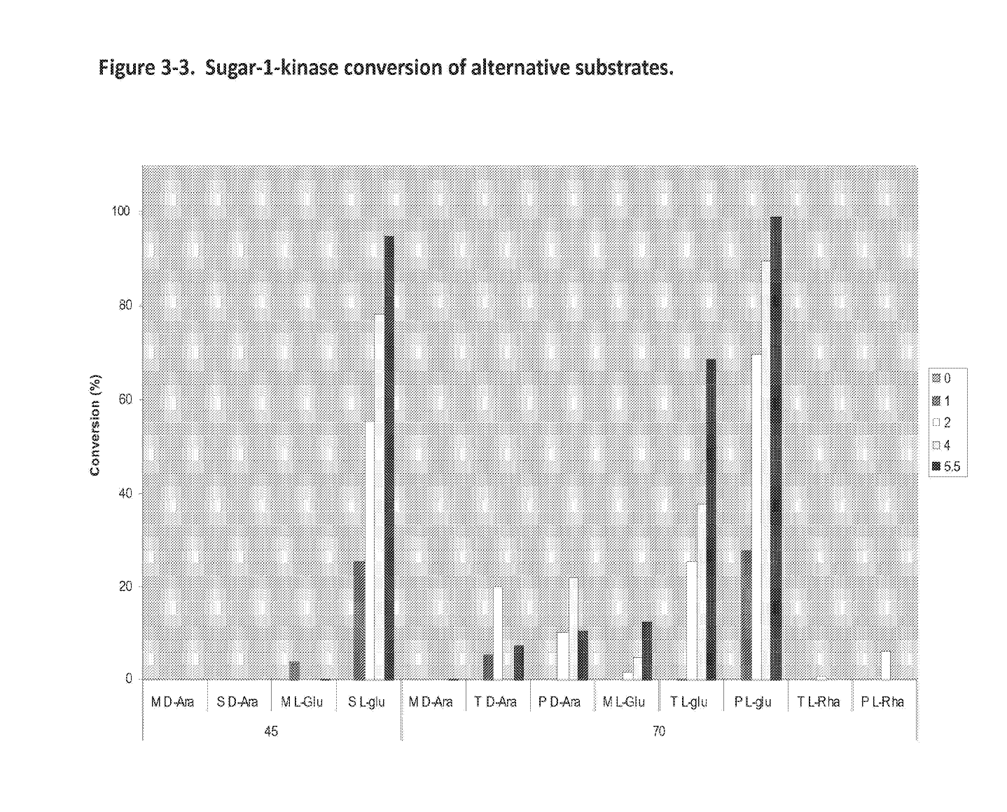

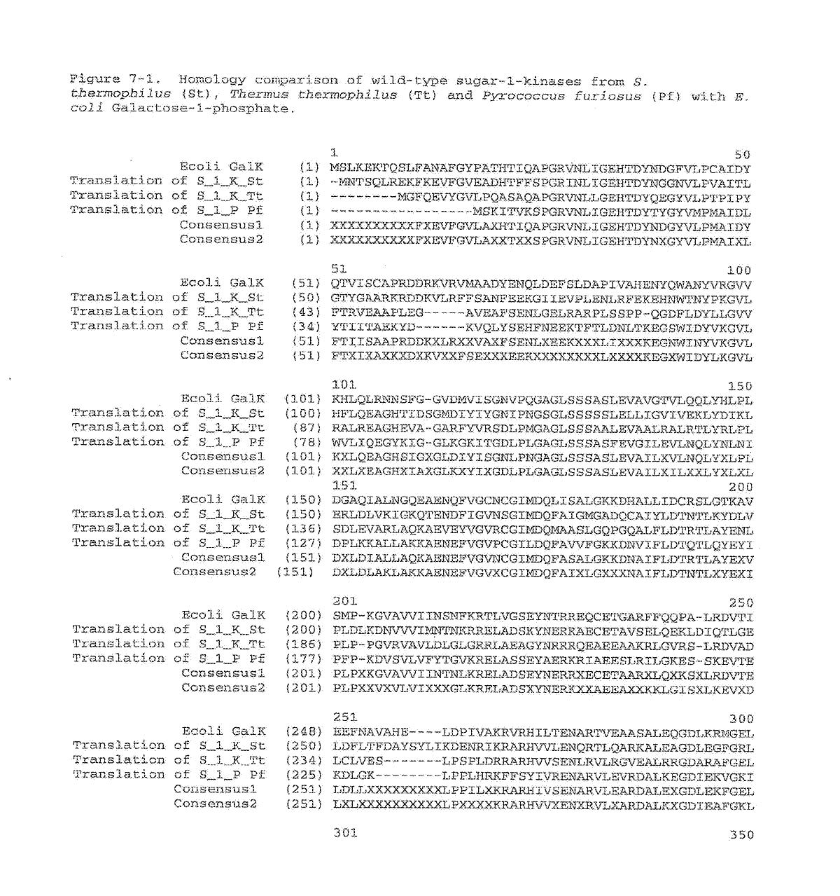

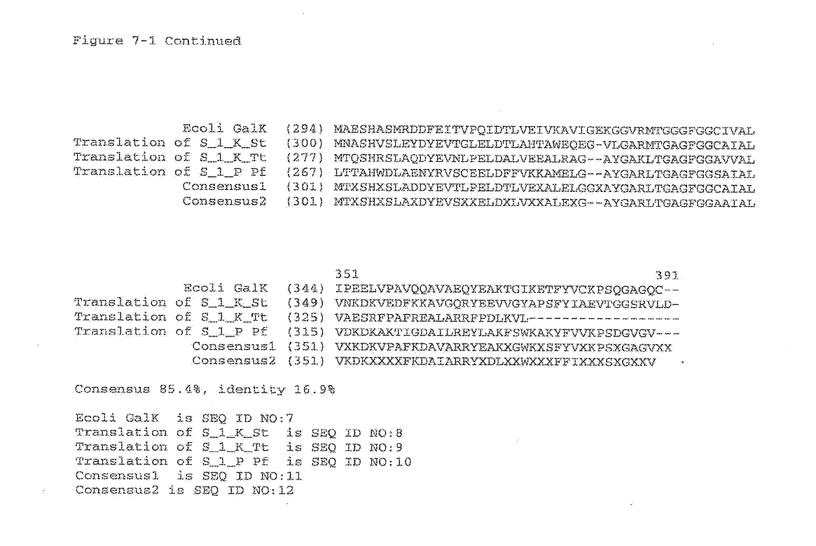

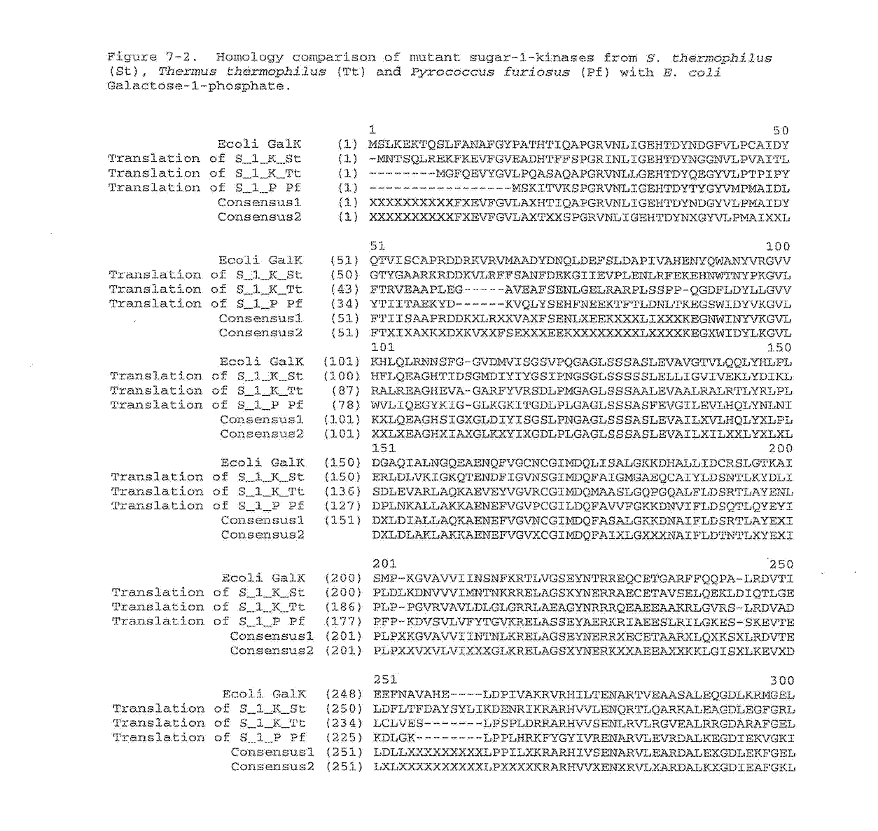

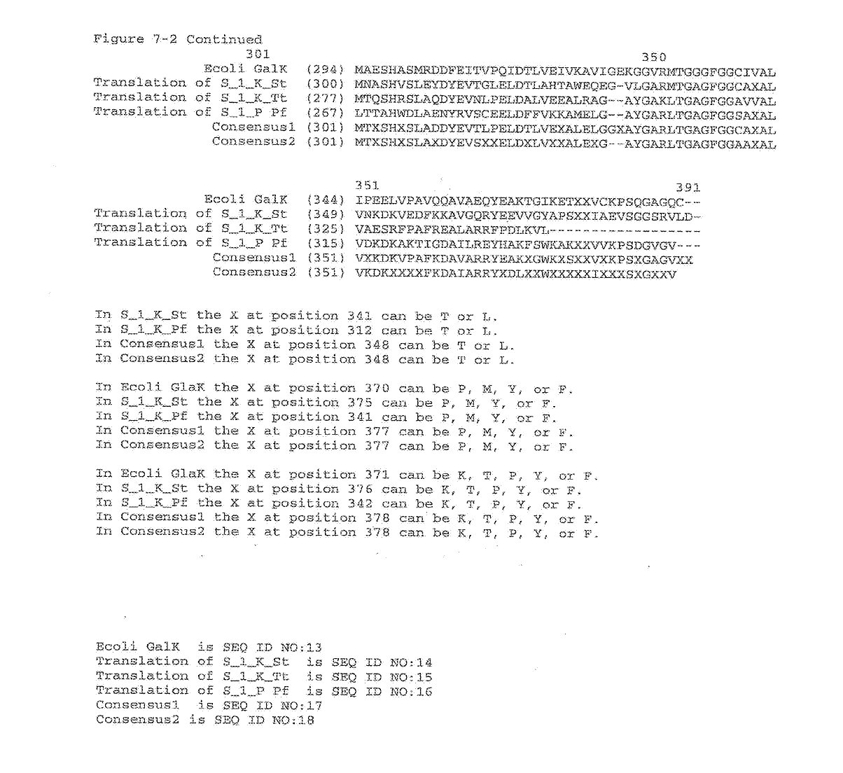

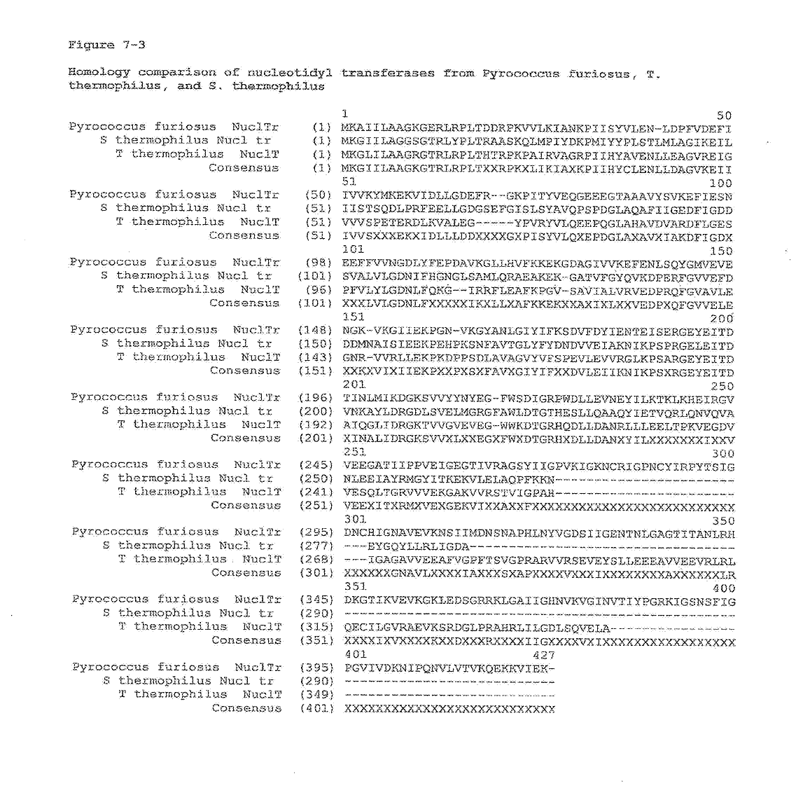

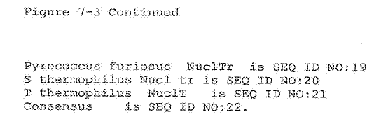

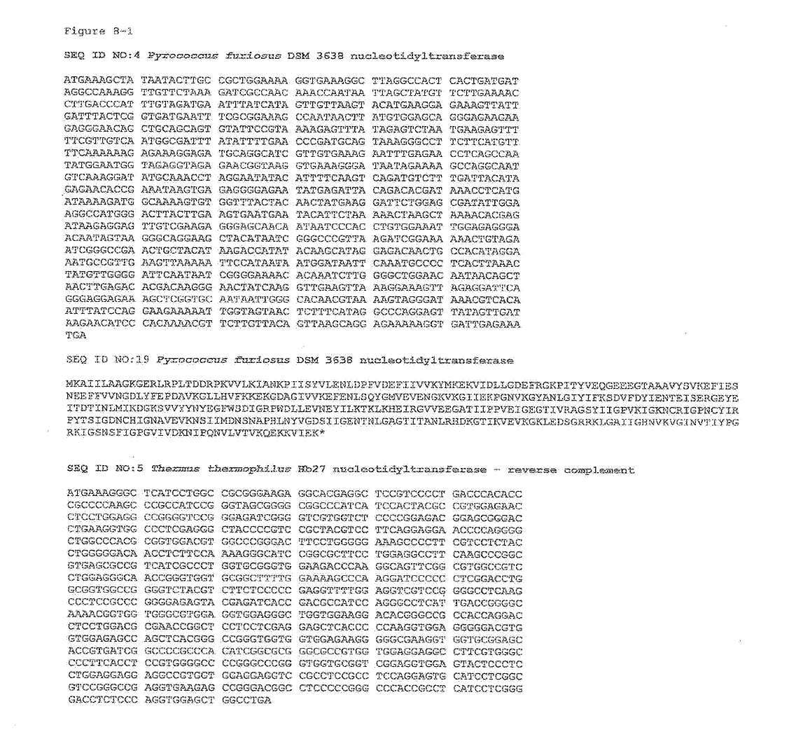

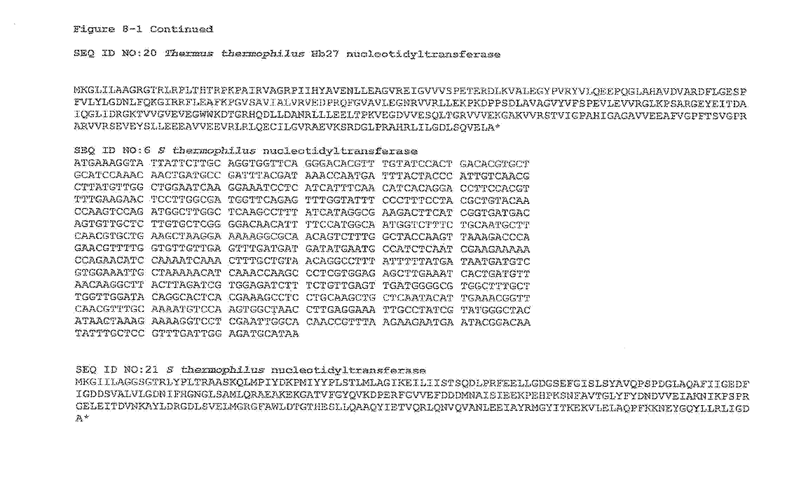

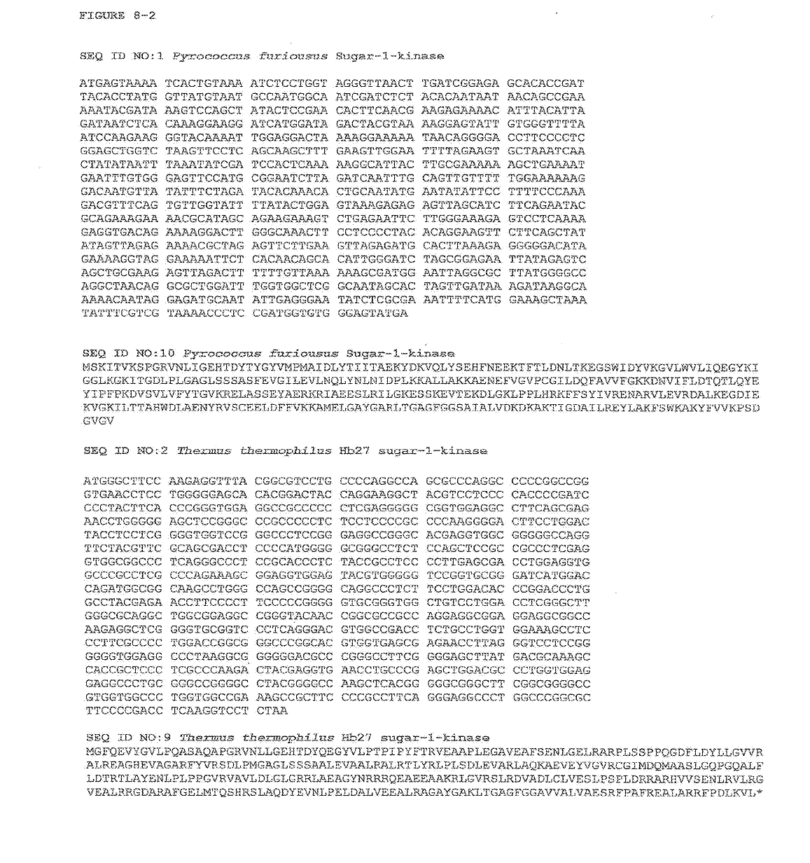

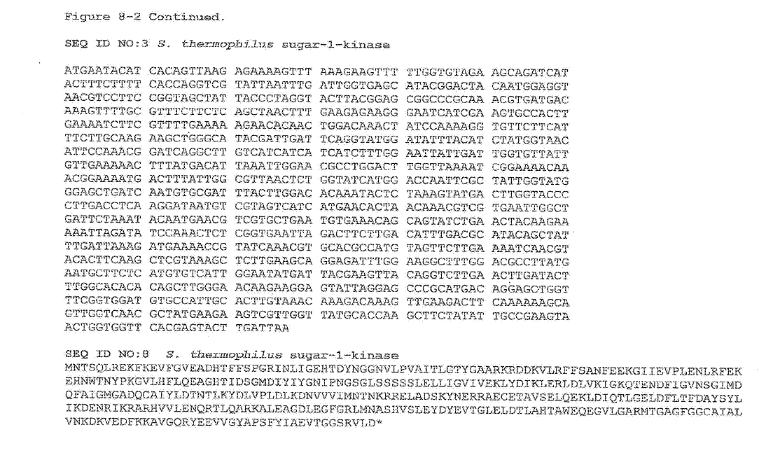

Also included in the invention are nucleotidyltransferase polypeptides, including SEQ ID NO:19-22. FIG. 7-3 shows the alignment of the nucleotidyltransferase polypeptides. Consensus (SEQ ID NO:22) is the alignment of the SEQ ID NOs:19, 20, and 21. There are several X's in the consensus sequence. In one embodiment of the invention, an X can stand for any amino acid. In other embodiment of the invention an X can stand for only the amino acids that occur in the corresponding position in SEQ ID NO:19, SEQ ID NO:20, and SEQ ID NO:21. For example, the X at position 19 of SEQ ID NO:22 can be D, R, or H in one embodiment.

The nucleotidyltansferases can form nucleoside-diphosphate (NDP) sugars by nucleotidyl transfer to any sugar-1-phosphate, such as D-sugar-1-phosphates or L-sugar-1-phosphates, such as D-galactose-1-phosphate, L-glucose-1-phosphate, L-rhamnose-1-phosphate, D-arabinose-1-phosphate, L-arabinose-1-phosphate, L-xylose-1-phosphate, D-xylose-1-phosphate, D-fucose-1-phosphate, L-fucose-1-phosphate, L-mannose-1-phosphate, D-mannose-1-phosphate, L-gulose-1-phosphate, 6-azido-D-galactose-1-phosphate, or a combination thereof. The nucleotidyltansferases can convert about 30, 40, 50, 60, 70, 80, 90, or 100% of the sugar-1-phosphate to its corresponding NDP sugar. TLC and inorganic phosphate assays (see example 5) can be used to test assay for activity.

Variant polypeptides that are at least about 80, 81, 82, 83, 84, 85, 86, 87, 88, 89, 90, 91, 92, 93, 94, 95, 96, 97, 98, or 99% identical to the sugar-1-kinase or nucleotidyltansferase polypeptides shown above, that retain sugar-1-kinase activity or nucleotidyltansferase activity are also polypeptides of the invention. Variant polypeptides can have one or more conservative amino acid variations or other minor modifications and retain biological activity, i.e., are biologically functional equivalents. A biologically active equivalent has substantially equivalent function when compared to the corresponding wild-type or mutant polypeptide. In one embodiment of the invention a polypeptide has about 1, 2, 3, 4, 5, 10, 15, 20, 30, 40, 50, or less conservative amino acid substitutions.

Percent sequence identity has an art recognized meaning and there are a number of methods to measure identity between two polypeptide or polynucleotide sequences. See, e.g., Lesk, Ed., Computational Molecular Biology, Oxford University Press, New York, (1988); Smith, Ed., Biocomputing: Informatics And Genome Projects, Academic Press, New York, (1993); Griffin & Griffin, Eds., Computer Analysis Of Sequence Data, Part I, Humana Press, New Jersey, (1994); von Heinje, Sequence Analysis In Molecular Biology, Academic Press, (1987); and Gribskov & Devereux, Eds., Sequence Analysis Primer, M Stockton Press, New York, (1991). Methods for aligning polynucleotides or polypeptides are codified in computer programs, including the GCG program package (Devereux et al., Nuc. Acids Res. 12:387 (1984)), BLASTP, BLASTN, FASTA (Atschul et al., J. Molec. Biol. 215:403 (1990)), and Bestfit program (Wisconsin Sequence Analysis Package, Version 8 for Unix, Genetics Computer Group, University Research Park, 575 Science Drive, Madison, Wis. 53711) which uses the local homology algorithm of Smith and Waterman (Adv. App. Math., 2:482-489 (1981)). For example, the computer program ALIGN which employs the FASTA algorithm can be used, with an affine gap search with a gap open penalty of −12 and a gap extension penalty of −2.

When using any of the sequence alignment programs to determine whether a particular sequence is, for instance, about 95% identical to a reference sequence, the parameters are set such that the percentage of identity is calculated over the full length of the reference polynucleotide and that gaps in identity of up to 5% of the total number of nucleotides in the reference polynucleotide are allowed.

Variant polypeptides can generally be identified by modifying one of the polypeptide sequences of the invention, and evaluating the properties of the modified polypeptide to determine if it is a biological equivalent. A variant is a biological equivalent if it reacts substantially the same as a polypeptide of the invention in an assay such as TLC assays or inorganic phosphate assays and 3,5-dinitrosalicylic assays, e.g. has 90-110% of the activity of the original polypeptide.

A conservative substitution is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

A polypeptide of the invention can further comprise a signal (or leader) sequence that co-translationally or post-translationally directs transfer of the protein. The polypeptide can also comprise a linker or other sequence for ease of synthesis, purification or identification of the polypeptide (e.g., poly-His), or to enhance binding of the polypeptide to a solid support. For example, a polypeptide can be conjugated to an immunoglobulin Fc region or bovine serum albumin.

Additionally, a polypeptide can be covalently or non-covalently linked to compounds or molecules other than amino acids such as indicator reagents. A polypeptide can be covalently or non-covalently linked to an amino acid spacer, an amino acid linker, a signal sequence, a stop transfer sequence, a transmembrane domain, a protein purification ligand, or a combination thereof. A polypeptide can also be linked to a moiety (i.e., a functional group that can be a polypeptide or other compound) that enhances an immune response (e.g., cytokines such as IL-2), a moiety that facilitates purification (e.g., affinity tags such as a six-histidine tag, trpE, glutathione, maltose binding protein), or a moiety that facilitates polypeptide stability (e.g., polyethylene glycol; amino terminus protecting groups such as acetyl, propyl, succinyl, benzyl, benzyloxycarbonyl or t-butyloxycarbonyl; carboxyl terminus protecting groups such as amide, methylamide, and ethylamide). In one embodiment of the invention a protein purification ligand can be one or more C amino acid residues at, for example, the amino terminus or carboxy terminus of a polypeptide of the invention. An amino acid spacer is a sequence of amino acids that are not associated with a polypeptide of the invention in nature. An amino acid spacer can comprise about 1, 5, 10, 20, 100, or 1,000 amino acids.

If desired, a polypeptide of the invention can be part of a fusion protein, which can also contain other amino acid sequences, such as amino acid linkers, amino acid spacers, signal sequences, TMR stop transfer sequences, transmembrane domains, as well as ligands useful in protein purification, such as glutathione-S-transferase, histidine tag, and Staphylococcal protein A, or combinations thereof. Other amino acid sequences can be present at the C or N terminus of a polypeptide of the invention to form a fusion protein. More than one polypeptide of the invention can be present in a fusion protein. Fragments of polypeptides of the invention can be present in a fusion protein of the invention. A fusion protein of the invention can comprise one or more polypeptides of the invention, fragments thereof, or combinations thereof.

A polypeptide of the invention can be produced recombinantly. A polynucleotide encoding a polypeptide of the invention can be introduced into a recombinant expression vector, which can be expressed in a suitable expression host cell system using techniques well known in the art. A variety of bacterial, yeast, plant, mammalian, and insect expression systems are available in the art and any such expression system can be used. Optionally, a polynucleotide encoding a polypeptide can be translated in a cell-free translation system. A polypeptide can also be chemically synthesized or obtained from bacteria cells that naturally produce the polypeptide.

### Polynucleotides

Polynucleotides of the invention contain less than an entire genome and can be single- or double-stranded nucleic acids. A polynucleotide can be RNA, DNA, cDNA, genomic DNA, chemically synthesized RNA or DNA or combinations thereof. The polynucleotides can be purified free of other components, such as proteins, lipids and other polynucleotides. For example, the polynucleotide can be 50%, 75%, 90%, 95%, 96%, 97%, 98%, 99%, or 100% purified. The polynucleotides of the invention encode the polypeptides of the invention described above. Polynucleotides of the invention can comprise other nucleotide sequences, such as sequences coding for linkers, signal sequences, TMR stop transfer sequences, transmembrane domains, or ligands useful in protein purification such as glutathione-S-transferase, histidine tag, and staphylococcal protein A.

Polynucleotides of the invention can be isolated. An isolated polynucleotide is a polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid sequences naturally found immediately flanking the recombinant DNA molecule in a naturally-occurring genome is removed or absent. Isolated polynucleotides also include non-naturally occurring nucleic acid molecules. A nucleic acid molecule existing among hundreds to millions of other nucleic acid molecules within, for example, cDNA or genomic libraries, or gel slices containing a genomic DNA restriction digest are not to be considered an isolated polynucleotide.

Polynucleotides of the invention can encode full-length polypeptides, polypeptide fragments, and variant or fusion polypeptides.

Degenerate nucleotide sequences encoding polypeptides of the invention, as well as homologous nucleotide sequences that are at least about 80, or about 90, 96, 98, or 99% identical to the polynucleotide sequences of the invention and the complements thereof are also polynucleotides of the invention. Percent sequence identity can be calculated as described in the “Polypeptides” section. Degenerate nucleotide sequences are polynucleotides that encode a polypeptide of the invention or fragments thereof, but differ in nucleic acid sequence from the wild-type polynucleotide sequence, due to the degeneracy of the genetic code. Complementary DNA (cDNA) molecules, species homologs, and variants of polynucleotides that encode biologically functional polypeptides of the invention also are polynucleotides of the invention. Polynucleotides of the invention can be isolated from nucleic acid sequences present in, for example, cell cultures. Polynucleotides can also be synthesized in the laboratory, for example, using an automatic synthesizer. An amplification method such as PCR can be used to amplify polynucleotides from either genomic DNA or cDNA encoding the polypeptides.

Polynucleotides of the invention can comprise coding sequences for naturally occurring polypeptides or can encode altered sequences that do not occur in nature. If desired, polynucleotides can be cloned into an expression vector comprising expression control elements, including for example, origins of replication, promoters, enhancers, or other regulatory elements that drive expression of the polynucleotides of the invention in host cells.

### Vectors and Host Cells

A polypeptide can be expressed in systems, e.g., cultured cells, which result in substantially the same post-translational modifications present as when the polypeptide is expressed in a native cell, or in systems that result in the alteration or omission of post-translational modifications, e.g., glycosylation or cleavage, present when expressed in a native cell.

Methods for preparing polynucleotides operably linked to an expression control sequence and expressing them in a host cell are well-known in the art. See, e.g., U.S. Pat. No. 4,366,246. A polynucleotide of the invention is operably linked when it is positioned adjacent to or close to one or more expression control elements, which direct transcription and/or translation of the polynucleotide.

An expression vector can be, for example, a plasmid, such as pBR322, pUC, or ColE1, or an adenovirus vector, such as an adenovirus Type 2 vector or Type 5 vector. Optionally, other vectors can be used, including but not limited to Sindbis virus, simian virus 40, alphavirus vectors, poxvirus vectors, and cytomegalovirus and retroviral vectors, such as murine sarcoma virus, mouse mammary tumor virus, Moloney murine leukemia virus, and Rous sarcoma virus. Minichromosomes such as MC and MC1, bacteriophages, phagemids, yeast artificial chromosomes, bacterial artificial chromosomes, virus particles, virus-like particles, cosmids (plasmids into which phage lambda cos sites have been inserted) and replicons (genetic elements that are capable of replication under their own control in a cell) can also be used. Polynucleotides in such vectors are preferably operably linked to a promoter, which is selected based on, e.g., the cell type in which expression is sought.

The expression vector can be transferred to a host cell by conventional techniques and the transfected cells are then cultured by conventional techniques to produce a polypeptide of the invention. The invention includes host cells containing polynucleotides encoding a polypeptide of the invention (e.g., a polypeptide, a fragment of a polypeptide, or variant thereof), operably linked to a heterologous promoter.

Host cells into which vectors, such as expression vectors, comprising polynucleotides of the invention can be introduced include, for example, prokaryotic cells (e.g., bacterial cells) and eukaryotic cells (e.g., yeast cells; fungal cells; plant cells; insect cells; and mammalian cells). Such host cells are available from a number of different sources that are known to those skilled in the art, e.g., the American Type Culture Collection (ATCC), Manassas, Va. Host cells into which the polynucleotides of the invention have been introduced, as well as their progeny, even if not identical to the parental cells, due to mutations, are included in the invention. Host cells can be transformed with the expression vectors to express the antibodies or antigen-binding fragments thereof.

One embodiment of the invention provides methods of producing a recombinant cell that expresses a polypeptide of the invention, comprising transfecting a cell with a vector comprising a polynucleotide of the invention. A polypeptide of the invention is then produced the recombinant host cell.

Isolation and purification of polypeptides produced in the systems described above can be carried out using conventional methods, appropriate for the particular system.

### Methods of Production of Sugar-1-Phosphates and Nucleoside-Diphosphate(NDP) Sugars

Sugar-1-kinases of the invention can be used to produce sugar-1-phosphates from sugars. One or more sugars are contacted with purified or partially purified one or more sugar-1-kinases of the invention such that the sugars are converted to the corresponding sugar-1-phosphates. ATP, MgCl₂, and phosphate buffer can be present in the reaction. The one or more sugars can be, for example, an L-sugar or a D-sugar such as D-galactose, L-galactose, L-glucose, D-glucose, D-glucoronate, L-rhamnose, D-arabinose, L-arabinose, L-xylose, D-xylose, L-ribose, D-ribose, D-fucose, D-fucose, L-fucose, L-xylose, L-Ixyose, D-xylose, L-mannose, D-mannose, L-gulose, 6-azido-D-galactose, or a combination thereof.

The reaction temperature for conversion of sugars to sugar-1-phosphates can be about 10, 20, 30, 45, 50, 55, 60, 70, 75, or 90° C.

The sugar-1-kinases can convert about 30, 40, 50, 60, 70, 80, 90, or 100% (or any range between about 30 and 100% conversion) of the sugar to its corresponding sugar-1-kinase. The sugar-1-kinases can complete this conversion in about 15, 30, 60 or less minutes, or about 1, 2, 3, 4, 5, 10, 24, 36, 48 or less hours (or any range between about 15 minutes and 48 hours).

The sugar-1-kinases of the invention can be thermostable at about 30, 45, 50, 55, 60, 70, 75, or 90° C. (or any range between about 30 and 90° C.) for about 10, 20, 30, 60, 75, 100, 120, 150 or more minutes (or any range between about 10 and 150 minutes). In one embodiment of the invention a sugar-1-kinase of the invention is thermostable for more than 10 minutes at 30, 60, or 75° C. Additionally, the sugar-1-kinases of the invention have a T₅₀ half-life at 30, 45, 50 or 60° C. for greater than 10, 20, 30, 40, 50, 60, or 120 minutes. The T₅₀ half-life and thermostablity of a sugar-1-kinase can be assayed using, for example a 3,5-dinitrosalicylic acid (DNS) assay.

Nucleotidyltransferases of the invention can be used to produce nucleoside-diphosphate (NDP) sugars from sugar-1-phosphates. One or more sugar-1-phosphates are contacted with purified or partially purified one or more nucleotidyltransferases of the invention such that the sugar-phosphates are converted to the corresponding nucleoside-diphosphate sugars. A nucleotide donor (such as UTP, dATP, dGTP, dTTP, dCTP), MgCl₂, pyrophosphatase (e.g., thermostable pyrophosphatase) can be present in the reaction. The one or more sugar-phosphates can be, for example, an L-sugar-1-phosphate or a D-sugar-1-phosphate such as D-galactose-1-phosphate, L-galactose-1-phosphate, L-glucose-1-phosphate, D-glucose-1-phosphate, D-glucoronate-1-phosphate, L-rhamnose-1-phosphate, D-arabinose-1-phosphate, L-arabinose-1-phosphate, L-xylose-1-phosphate, D-xylose-1-phosphate, L-ribose-1-phosphate, D-ribose-1-phosphate, D-fucose-1-phosphate, D-fucose-1-phosphate, L-fucose-1-phosphate, L-xylose-1-phosphate, L-Ixyose-1-phosphate, D-xylose-1-phosphate, L-mannose-1-phosphate, D-mannose-1-phosphate, L-gulose-1-phosphate, 6-azido-D-galactose-1-phosphate, or a combination thereof.

The reaction temperature for conversion of sugar-1-phosphates to NDP sugars can be about 10, 20, 30, 45, 50, 55, 60, 70, 75, or 90° C.

The nucleotidyltransferases can convert about 30, 40, 50, 60, 70, 80, 90, or 100% (or any range between about 30 and 100% conversion) of the sugar-1-phosphate to its corresponding NDP sugar. The nucleotidyltransferases can complete this conversion in about 15, 30, 60 or less minutes, or about 1, 2, 3, 4, 5, 10, 24, 36, 48 or less hours (or any range between about 15 minutes and 48 hours).

The nucleotidyltransferases of the invention can be thermostable at about 30, 45, 50, 55, 60, 70, 75, or 90° C. (or any range between about 30 and 90° C.) for about 10, 20, 30, 60, 75, 100, 120, 150 or more minutes (or any range between about 10 and 150 minutes). In one embodiment of the invention a nucleotidyltransferase of the invention is thermostable for more than 10 minutes at 30, 60, or 75° C. Additionally, the nucleotidyltransferases of the invention have a T₅₀ half-life at 30, 45, 50 or 60 ° C. for greater than 10, 20, 30, 40, 50, 60, or 120 minutes. The T₅₀ half-life and thermostablity of a nucleotidyltransferase can be assayed using, for example a TLC assay or an inorganic phosphate assay using a malachite green molybdenum complex and a thermophilic pyrophosphatase.

In one embodiment of the invention, one or more sugars can be contacted with one or more sugar-1-kinases and one or more nucleotidyltransferase under reaction conditions wherein one or more sugars are converted to NDP sugars. The sugar-1-kinases and nucleotidyltransferases can convert about 30, 40, 50, 60, 70, 80, 90, or 100% (or any range between about 30 and 100% conversion) of the sugar to a corresponding NDP sugar. The sugar-1-kinases and nucleotidyltransferases can complete this conversion in about 15, 30, 60 or less minutes, or about 1, 2, 3, 4, 5, 10, 24, 36, 48 or less hours (or any range between about 15 minutes and 48 hours). The sugar-1-kinases and nucleotidyltransferases can be added to the reaction at the same time, or alternatively, the sugar-1-kinases can be added and then the nucleotidyltransferases can be added at a later time (e.g., 5, 10, 20, 30, 40, 60, 120 or more minutes after the sugar-1-kinase is added).

One or more glycosyltransferases can be added to a NDP sugar reaction of the invention to glycosylate the NDP sugar or to attach the NDP sugar to one or more types of aglycones.

### EXAMPLES

### Example 1

### Assays for Sugar-1-kinase Activity

The formation of a phosphorylated sugar by kinase activity can be monitored by a number of methods. One method for detecting sugar-1-kinase activity is the 3,5-dinitrosalicylic acid (DNS) assay. This assay exploits the fact that reducing sugars can reduce compounds such as 3,5-dinitrosalicylic acid, which undergo a color change upon reduction. This assay can be used for sugar-1-kinases since the product of their reaction (sugar-1-phosphate) no longer has the ability to reduce DNS. Therefore, when the reaction is complete no color change occurs when incubated with DNS and the result is a yellow color. However, when reducing sugar remains, the result is reduction of DNS and red/brown color. This assay is furthermore concentration dependent providing a linear color change from 0.1 to 10 mM reducing sugar.

As displayed in FIG. 1-1, the DNS assay was applied in 96-well format and is extremely useful in methods such as protein engineering where it can be used as a high-throughput screen. For directed evolution, cells were grown, induced, and lysed in 96 well plates. The cell lysate was then incubated with ATP, MgCl₂, and the sugar substrate of interest. Following this incubation, DNS reagent was added to each well of the 96-well plate and incubated at 95° C. in a PCR block. The resulting wells were sorted by color and wells with less color than the positive controls (FIG. 1-1) were selected as hits with better activity. Additionally, this assay was used to track sugar-1-kinase reaction versus time and to see the extent of reaction as detailed in Example 3.

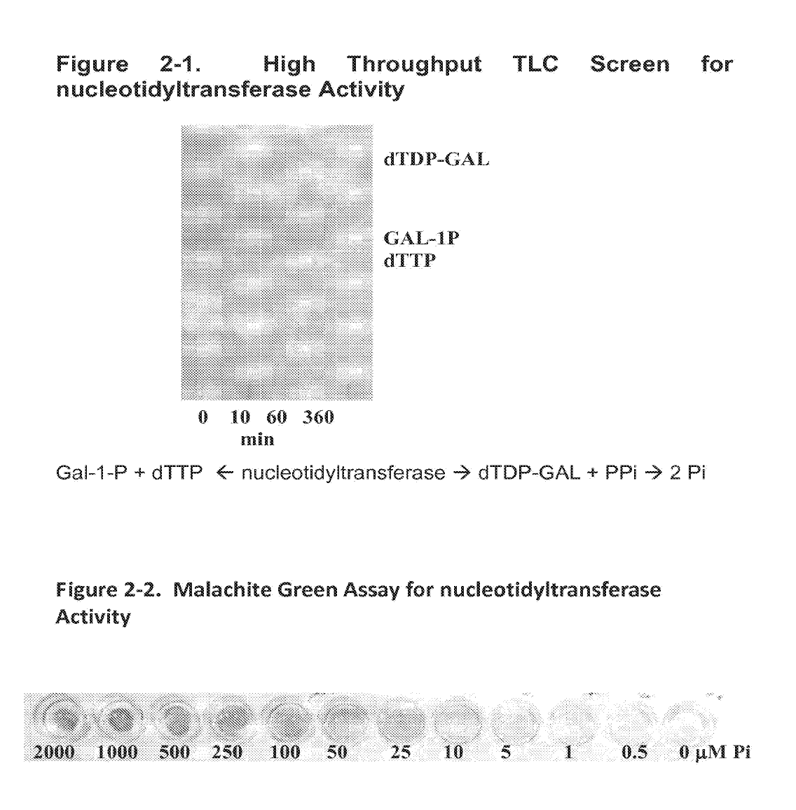

Thin Layer Chromatography (TLC) also proved vital to detection of reaction products. The best system was determined to be a mobile phase of 1:1 isopropyl alcohol to concentrated ammonia with a solid phase of silica gel. Staining was typically achieved with KMnO₄. FIG. 1-2 displays the separation and staining of standard of D-galactose, ATP, and Galactose-1-phosphate. High-performance liquid chromatography (HPLC) can also be used to detect reaction products.

### Example 2

### Nucleotidyltransferase Assay

In order to test nucleotidyltransferase enzyme activity, which forms a NDP-sugar from a phosphorylated sugar and a nucleotide triphosphate, a convenient method for reaction analysis was first desired. Many methods exist to monitor the reaction by HPLC and LC-MS as the workhorse assay method. However, these assays are laborious and tedious and utilize expensive equipment. They are also not suitable for a high-throughput screening assay required in a directed evolution protein engineering experiment. We therefore developed 2 new assays methods.

The first is based on TLC using the same conditions as the sugar-1-kinase TLC assay (FIG. 2-1). This is convenient because it allows us to track the coupled reaction of sugar-1-kinase and nucleotidyltransferase by a single method. Additionally, TLC allows the rapid analysis of multiple samples with much higher throughput that HPLC. Finally, prep-TLC can facilitate purification of 25-50 mg of NDP-sugars.

The second assay developed for nucleotidyltransferase activity is an adaptation of an inorganic phosphate assay using a malachite green molybdenum complex and a thermophilic pyrophosphatase. A solution of 300 mL water, 60 mL H₂SO₄, 0.44 g Malachite green pyrophosphatase and the test solution was prepared. Directly prior to use, 10 mL malachite green solution is mixed with 2.5 mL 7.5% (w/v) ammonium molybdate and 0.2 mL TWEEN®20 (polysorbate)(11% w/v). The resulting solution is an orange color. In the presence of phosphate a blue/green color rapidly develops. The assay is sensitive from 1 μM to 100 μM inorganic phosphate as displayed in FIG. 2-2 and is interfered with very little by other compounds. This assay can be used to analyze nucleotidyltransferase activity since the by-product is pyrophosphate, which can be readily converted to two molecules of phosphate by pyrophosphatase.

Therefore, nucleotidyltransferase activity can be assayed by mixing the test nucleotidyltransferase solution with malachite green and pyrophosphatase in an appropriate buffer solution. About 1 μl of a 2000 u/ml concentration pyrophosphatase per 100 μl of reaction can be used.

### Example 3

### Cloning and Characterizing Thermostable Sugar-1-Kinase Genes

In order to identify an enzyme suitable for large scale production of phosphorylated sugars in an industrial environment we wanted to circumvent the problem with stability by indentifying a thermostable enzyme that could be used. There were two challenges that needed to be overcome to find a suitable thermostable enzyme to use. First, thermostable enzymes are not always expressed well in a mesophile like E. coli due to folding, codon usage and other issues. Second, enzymes isolated from the three main classes of thermophilic organisms (hyperthermophile, extreme thermophile, and moderate thermophile) often have varying levels of expression issues, varying levels of thermostability and thermotolerance, and varying minimal temperatures for activity (which would be important in employing the enzyme in an industrial setting). Enzymes were selected in order to test the level of expression and activity from examples of each class of thermophiles.

Thus sugar-1-kinase genes were cloned from three representative thermophiles: Pyrococcus furiosus (a hyperthermophile)—SEQ ID NO:1; Thermus thermophilus (an extreme thermophile) SEQ ID NO:2; and Streptococcus thermophilus (a moderate thermophile) SEQ ID NO:3. Genomic DNA was prepared, specific primers designed, and the genes were amplified by PCR and cloned into a plasmid under the control of T7 Promoter as N-terminally 6-His tagged fusions. Correct constructs of each gene were obtained as verified by sequencing and restriction analysis.

The sugar kinase proteins were expressed recombinantly in E. coli induced with 0.5mM IPTG and partially purified cell lysates were then assayed (100 μL) with 400 μmM ATP, 3.5 mM Mg²⁺, and 8 mM D-galactose at three different temperatures, 37, 45, and 55° C. Samples were taken at different time points and analyzed by our developed DNS reducing sugar assay, with the results displayed in FIG. 3-1. A negative control was treated similarly and consisted of the host strain with empty plasmid.

Of the three different sugar kinases, the enzyme from S. thermophilus (Sugar-1-kinase-5) had the most activity in partially purified cell extract at 37° C., whereas the T. thermophilus (Sugar-1-kinase-T) and P. furiousus (Sugar-1-kinase-P) enzymes both appeared to be more active at temperatures higher than 37° C. This result demonstrated that all of the enzymes were actively expressed in E. coli and furthermore were active at temperatures as high as 55° C.

The thermostabilities of all three thermophilic sugar-1-kinases were investigated and compared to the E. coli GalKMLYH mutant by incubating 100 μL of partially purified cell extract at various temperatures and then assaying the enzymes as above. The results (Table 1) demonstrated that all of the thermophilic enzymes possessed very high stability at 30° C. and a range of stability at elevated temperatures as high at 90° C. The most stable enzyme tested was clearly Sugar-1-kinase-P which maintained activity at temperatures as high as 90° C. for one hour, yet still displayed activity at lower temperatures. Production of D-Galactose-1-phosphate as the reaction product from D-galactose and ATP was confirmed by HPLC and TLC using authentic Galactose-1-phosphate.

TABLE 1
Thermostability T₅₀ of Sugar Kinases

| | 30° C. | 60° C. | 75° C. | 90° C. |
|---|---|---|---|---|
| E. coli specificity | 10 min | 0 min | 0 min | 0 min |
| mutant | | | | |
| S. thermophilus | >120 min | 10 min | 0 min | 0 min |
| T. thermophilus | >120 min | >120 min | 60 min | 10 min |
| P. furiousus | >120 min | >120 min | >120 min | 60 min |

Wth enzymes in hand with much greater stability, substrate specificity on a variety of D- and L-sugars was tested with each enzyme. Partially purified Sugar-1-kinase was incubated with D-arabinose, L-arabinose, D-glucose, L-glucose, D-ribose, L-ribose, D-fucose, L-fucose, D-galactose, D-glucuronate, L-gulose, L-rhamnose, L-Ixyose, and D-xylose in the presence of Mg²⁺ and ATP at both 45° C. and 75° C. The results as shown in FIG. 3-2, suggested that D-galactose is the natural substrate for each of these sugar kinases, and that L-glucose is a substrate to a lesser degree. The results also suggested that to some degree D-arabinose and L-Rhamnose might be substrates for these enzymes. A time course assay was utilized to further analyze, to what degree L-glucose, D-arabinose, and L-rhamnose could be converted by each of the three enzymes. As displayed in FIG. 3-3, L-glucose appeared to be a good alternative substrate and Sugar-1-kinase-P seemed to convert it the best.

These results suggest that several substrates were converted by the enzymes without any substrate engineering. In particular, the L-glucose reaction proceeded to 95-100% completion for Sugar-1-kinase-S at 45° C. and at 70° C. for Sugar-1-kinase-P in ˜300 minutes using only partially purified cell extract. It is notable that both Sugar-1-kinase-P and Sugar-1-kinase-S had better productivity and conversion with L-glucose using small amounts of partially purified protein than the engineered E. coli mutant (Sugar-1-kinaseMLYH) had using high concentrations of purified protein.

### Example 4

### Improving Specificity of Thermostable Kinases

With the stability issues and commercial viability for the sugar kinases solved, the next issue was to test the substrate specificity of the sugar kinases.

Due to the apparent promiscuity identified in the Sugar-1-kinase-P and Sugar-1-kinase-S enzymes, more than sufficient stability, and high activity in cell lysates, these enzymes were chosen as models for further engineering. The high-throughput screen using the DNS reducing sugar assay described in Example 1 was optimized and was applied to directed evolution for more promiscuous Sugar-1-kinase enzymes. First, a library of Sugar-1-kinase genes was created using error-prone PCR, cloned into the expression vector and transformed into E. coli to create a library of 1×10⁴ clones expressing mutant Sugar-1-kinase enzymes. The library was analyzed for mutation rate by sequencing and activity. The mutation rate was such that the average number (n=10) of base pair changes was approximately 4. The number of mutants with significantly lower activity than the WT was determined to compose 80% of the library.

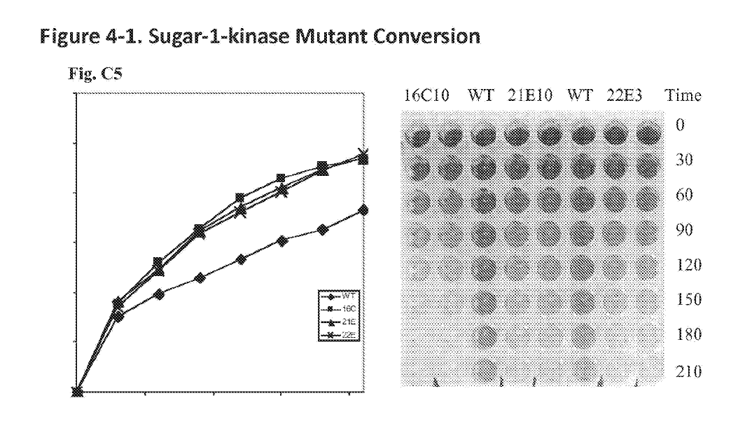

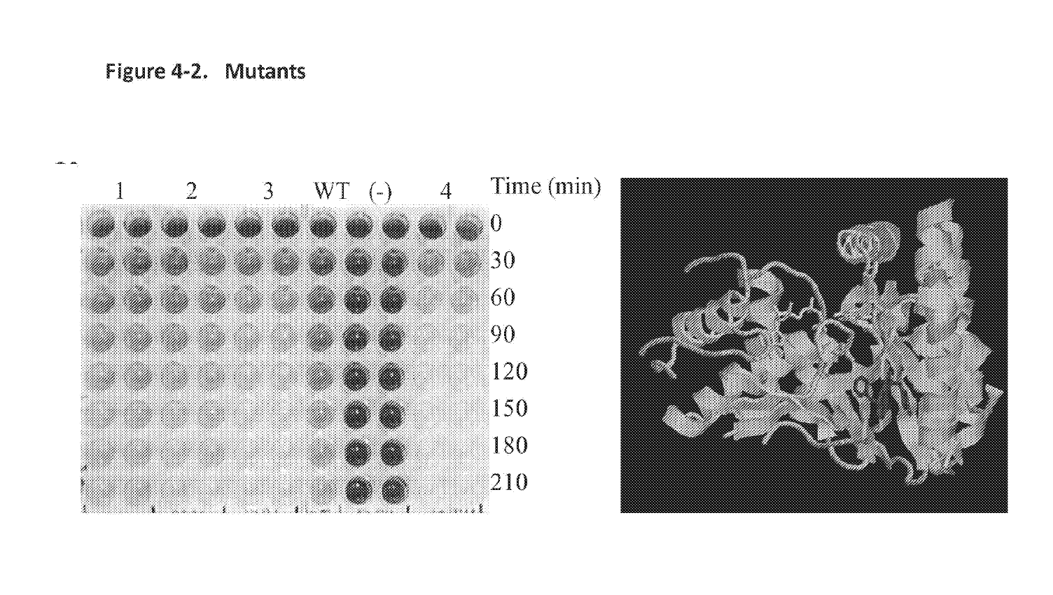

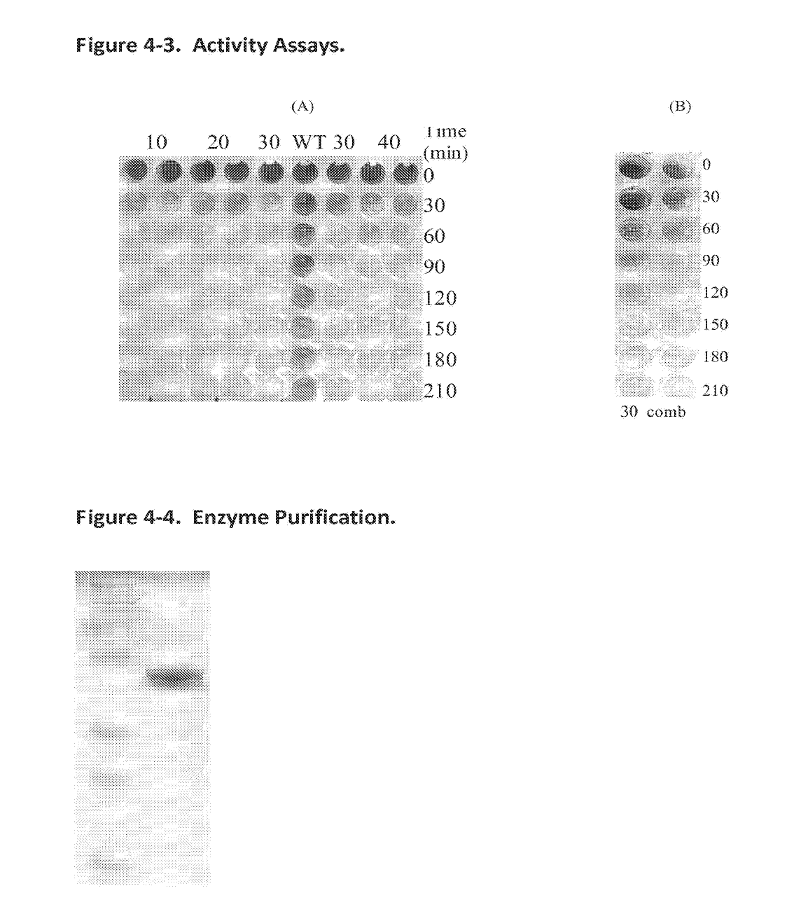

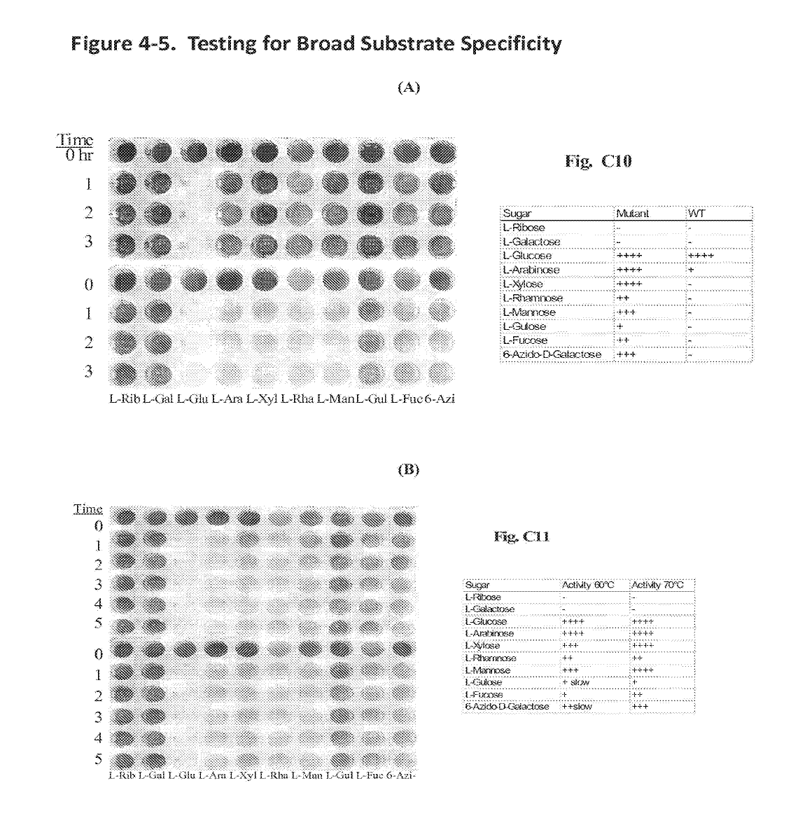

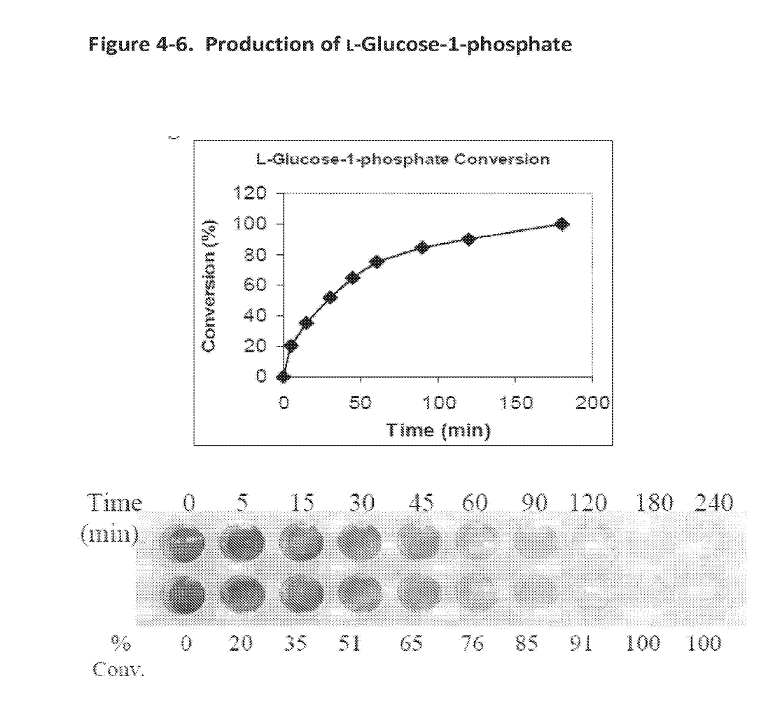

The library members were picked into 96 well plates, grown, expression induced, pelleted, lysed, and the cell extract was assayed with L-glucose as the substrate. Upon sorting of the Sugar-1-kinase-S library on L-glucose 3 improved mutants were identified that could convert L-glucose with an improved rate of approximately 2-fold. These mutants were named 16C10, 21E10, and 22E3 (See Table 2). FIG. 4-1 displays on time course assay of the isolated mutants compared with WT Sugar-1-kinase-S using L-glucose as a substrate. The mutants were sequenced and there were no conserved mutations among the three mutants. Therefore these mutants may be combined in the future to further improve the activity.

Upon sorting a similar sized random library of Sugar-1-kinase-P, ten mutants were identified with improved ability to convert L-glucose. The four best of those ten mutants were selected and compared to WT Sugar-1-kinase-P using L-glucose as a substrate as displayed in FIG. 4-2. These mutants were between 3-5 fold improved over the WT enzyme. The two best of these mutants (Mutant 26 and Mutant 27 shown in column 3 and 4 of FIG. 4-2 respectively) were sequenced (see Table 2) and it was determined that while no mutations were

TABLE 2
Mutations

| Gene Source | Mutant Name | Orgin of Mutations | Amino Acid Substitution(s) |
|---|---|---|---|
| S. Thermophilus | 16C20 | Error Prone PCR | N120S, D183E, T191S, Y376F, T381S |
| | 21E10 | Error Prone PCR | E71D, V199I |
| | 22E3 | Error Prone PCR | D221G |
| P. Furiousus | 26 | Error Prone PCR | N119H, K130N, S239G, F238Y, I312L |
| | 27 | Error Prone PCR | I312T, L332H |
| | 30 | Saturation Mutation | Y341P, F342K |
| | 32 | Error Prone PCR | Y341M, F342T |
| | PK-27 | Site directed mutagenesis | I312T, L332H, Y341P, F342K |

conserved, there was a high mutation frequency near the C-terminus (FIG. 4-2) of the protein. Since the crystal structure of Sugar-1-kinase-P had been previously solved, some insight could be made into the effect of these mutations. Most of the mutations occurred far from the active site (ADP and galactose, FIG. 4-2). However, the C-terminus seemed to help form the shape of the active site and it was thus hypothesized that these mutations were disruptive of the active site shape making the active site more accessible to unnatural substrates. We used this hypothesis to create a semi-rational library of Sugar-1-kinase-P mutants at amino acid positions 341 and 342. These positions are near the C-terminus and appeared to make large contributions to the active site shape. Thus, saturation mutagenesis was performed on both residues simultaneously, swapping the natural residues out with all 19 other possible amino acids. This semi-rational library was screened for improved activity on L-glucose and approximately 30% of the library had significantly improved activity, thus confirming the hypothesis. The best four mutants were selected and subjected to a time course reaction with L-glucose as the substrate and compared to WT Sugar-1-kinase-P. All four mutants were approximately 10-fold better than the WT as displayed in FIG. 4-3A and the two best (Mutant 30 and Mutant 32) were sequenced (see Table 2).

At this point Sugar-1-kinase-P mutants had been created and isolated that had activities on L-glucose that were impressively 3-10 fold better than the WT enzyme. The best mutant for each methodology was subsequently selected and PCR overlap extension was utilized to combine the mutations of each into a single construct. This single construct was successfully created (PK-27) and had 4 amino acid mutations as described in Table 2. This mutant was compared to the best Sugar-1-kinase-P mutant in a time course assay with L-glucose. The combined mutant (Sugar-1-kinase-PK27) performed better than the best round 1 mutant by approximately 3-fold (FIG. 4-3B), thus this mutant could convert L-glucose to L-glucose-1-phosphate approximately 30-fold better and WT Sugar-1-kinase-P.

The combined mutant enzyme was purified using IMAC making use of the 6-His tag. 1.6 L of E. coli culture was grown and induced, followed by cell lysis. A 6 mL Co²⁺ resin column was utilized to purify 60 mg of enzyme at 8.6 mg/ml. SDS-PAGE showed the protein to be of expected size and apparently homogeneous (FIG. 4-4). Due to the thermostable nature of the protein, it could additionally be purified to near homogeneity by simply lysing expressed cells, followed by heat denaturation of the endogenous proteins and filtration.

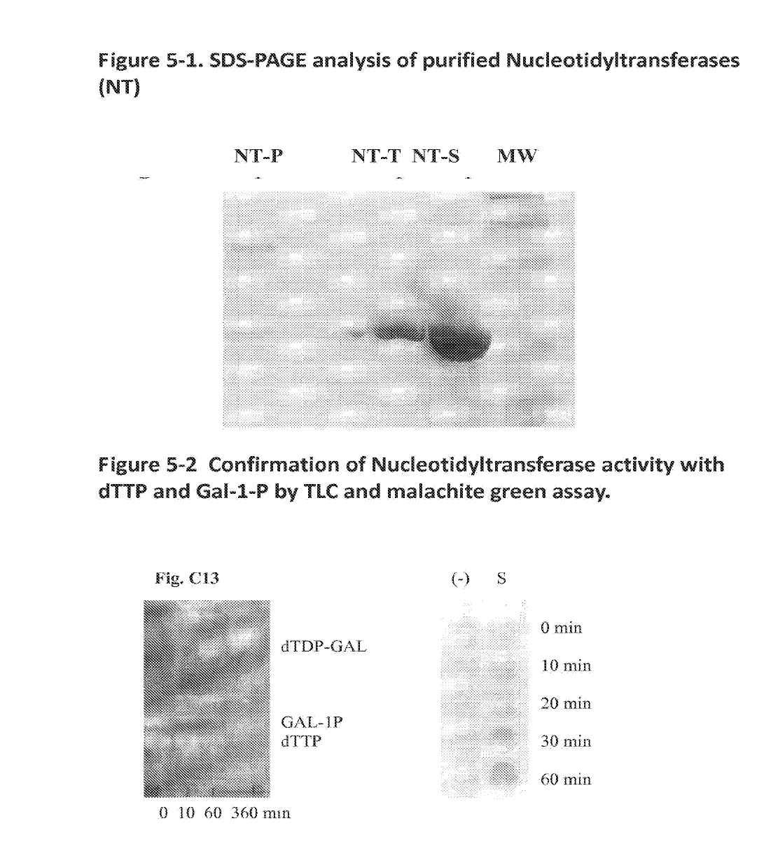

Often, when applying protein engineering to activity on a new substrate the resulting enzyme has relaxed substrate specificity which we wanted to achieve. The purified Sugar-1-kinase-PK27 was then tested for the conversion of a variety of sugars and compared to purified WT Sugar-1-kinase-P. The reactions were setup with 8 mM of different sugars (L-ribose, L-galactose, L-glucose, L-arabinose, L-xylose, L-rhamnose, L-mannose, L-gulose, L-fucose, and 6-Azido-D-galactose), 2.4 mg/ml enzyme, 12 mM ATP, and 5 mM MgCl₂ in pH 7.5 phosphate buffer. Samples were taken every hour and analyzed by DNS assay (FIG. 4-5A). The results were very clear, while the WT Sugar-1-kinase-P only displayed activity on L-glucose, the substrate specificity had been significantly broadened for Sugar-1-kinase-PK27. Greater than 75% conversion was achieved for L-glucose, L-arabinose, L-xylose, L-rhamnose, L-mannose, and 6-Azido-D-galactose. Additionally, ˜50% conversion was displayed with L-gulose and L-fucose as substrates.

The stability and activity of the Sugar-1-kinase-PK27 was measured to make sure similar problems with stability were not created by the mutations. The substrate specificity assay was repeated at different temps (60, 70, and 80° C.) as displayed in FIG. 4-5B. At 80° C. a significant amount of protein precipitation was observed, and activity was not very high. However, at 60° C. and 70° C. the enzyme did not precipitate and appears to have optimum activity around the 70° C. range.

In summary, while the original GalKYMLH mutant was neither active on L-sugars, nor stable enough for industrial utilization, we succeeded in developing a new Sugar-1-kinase with broad activity towards L-sugar substrates and very high thermostability that can be readily purified and handled. We successfully demonstrated that the enzyme could convert >75% of a variety of L-sugars and 6-azido-D-galactose.

Gram scale synthesis of L-sugar-1-phosphates has been demonstrated. A reaction containing 0.2 g/L Sugar-1-kinase-PK27, 92 mM L-glucose, 100 mM ATP, 5 mM MgCl₂ in 40 mL pH 7.5 phosphate buffer was incubated at 70° C. Samples were taken and analyzed by DNS assay to determine the extent of reaction as displayed in FIG. 4-6. The reaction reached 100% in just 3 hours producing 1 gram of L-glucose-1-phosphate with a very high space-time yield of 200 g/L*d. This is the first commercially viable system for the enzymatic production of L-sugar-1-phosphates.

Additionally, production of D-galactose-1-phosphate was carried out on 100 mg scale using only partially purified cell extract from 5 mL culture of E. coli expressing Sugar-1-kinase-P. A 4.5 mL mixture of 110 mM D-galactose, 130 mM ATP, and 3.5 mM MgCl2 was mixed with a one tenth volume of cell extract and incubated at 70° C. Using this crude system, 100 mg of D-galactose was converted to 144 mg D-galactose-1-Phosphate in 2 hours for a space time yield of 384 g/L*d.

The reaction of sugars with the wild type and mutant sugar-1-kinse such as those from Pyrococcus furiousus can also be monitored by following ATP consumption in the reaction. The amount of ATP consumption directly correlates with the amount of sugar-1-kinase produced. For example, to produce additional sugar-1-phosphates a series of experiments were carried out as follows, In a reaction mix containing 50 mM sodium phosphate buffer at pH 7.5, 100 mM ATP, 200 mM of the sugar being tested, 5mM MgC12 either 1 ug/ml of either the PK27 mutant or wild-type P. furiosus enzyme were added. The reaction was incubated at 60° C. for 20 hours. ATP and ADP concentrations were analyzed by HPLC using a Supelcosil LC-18-T column with a flow rate of 1.0 mL/min of 0.05 M. KH₂P0₄/4 mM tetrabutylammonium hydrogen sulfate and a linear gradient solvent program of 0-30% methanol over 30 min. The percent conversion of ATP to ADP was calculated. Sugar-1-phosphate was analyzed by HPLC using Supelcosil LC-SAX column 0.05 M K-phosphate buffer, pH 6.0

GalK activity by ATp to ADp conversion, 20 h. Percentages indicate degree reaction proceeded to completion within 20 hours.

| | | Mutant | WT |
|---|---|---|---|
| Sugar used | Sugar phosphate produced | PK27 | Pyrococcus furiosus |
| D-Galactose | D-Galactose-1-phsophate | 100% | 90% |
| D-fucose | D-fucose-1-phsophate | 74% | 78% |
| L-fucose | L-fucose-1-phsophate | 40% | 40% |
| D-mannose | D-mannose-1-phsophate | 70% | 54% |
| D-xylose | D-xylose-1-phsophate | 64% | 50% |

### Example 5

### Coupling of Sugar-1-kinase-Nucleotidyltransferase Enzyme Activities

In order to produce sugar nucleotides, we attempted to couple the broad specificity Sugar-1-kinase with the previously created variant of the nucleotidyltransferase from Salmonella enterica [49]. This enzyme was previously created using rational protein engineering based on a solved crystal structure. While the natural substrate for this nucleotidyltransferase is D-glucose, the variant nucleotidyltransferase has been show to convert a variety of sugar-1-phosphates to NDP-sugars with varying degrees of conversion. However, similar to our attempts to utilize the E. coli Sugar-1-kinase, this enzyme also had significant issues with stability and did not have the ability to convert any L-sugar-1-phosphates to corresponding NDP-L-sugars.

We then cloned the nucleotidyltransferase homologs from each of the three thermophiles: Pyrococcus furiosus (a hyperthermophile) SEQ ID NO:4; Thermus thermophilus (an extreme thermophile) SEQ ID NO:5, and Streptococcus thermophilus (a moderate thermophile) SEQ ID NO:6. However, there were no known nucleotidyltransferase genes from T. thermophilus and S. thermophilus, so homologs of unknown activity were chosen. The use of thermophilic enzymes would resolve the stability concerns and additionally allow high temperature simultaneous reaction with Sugar-1-kinase-PK27. Therefore, genomic DNA was prepared, specific primers designed, and the genes were amplified by PCR and cloned into a plasmid under the control of T7 Promoter as N-terminally 6-His tagged fusions. Correct constructs of each gene were obtained as verified by sequencing and restriction analysis.

The nucleotidyltransferase proteins were expressed recombinantly in E. coli induced with 0.5mM IPTG and purified using Co²⁺ IMAC. The purified proteins were compared by SDS-PAGE analysis. Nucleotidyltransferase-P was expressed in E. coli, although poorly. Both nucleotidyltransferase-T and nucleotidyltransferase-S were expressed very well in E. coli. The activity of all three enzymes were tested using a malachite green assay. To run this test, a malachite green Assay Solution was made containing 405 μl of 15 mM Glucose-1-phospate in water, 405 μl of 15 mM dTTP in HEPES buffer, 4.5 μl 1M MgCl₂, and 5 μl of thermostable inorganic pyrophosphatase (New England Biolabs).

Then 800 μl of this malachite green Assay Solution was mixed with 3.2 ml HEPES buffer. 99 μl of the resulting mixture was then distributed into different tubes and 1 μl of desalted enzyme prepared from a shake flask fermentation was added to each tube. All three enzymes showed significant activity using this malachite green assay at 50° C. The nucleotidyltransferase-S was further analyzed. Approximately 90 mg of nucleotidyltransferase-S was purified from 1.6 L of E. coli cell culture and was concentrated to approximately 11.6 mg/ml. An SDS-PAGE analysis of purified nucleotidyltransferase is shown in FIG. 5-1.

The nucleotidyltransferase-S enzyme was chosen for further study due to its high expression in E. coli. Nucleotidyltransferase activity was measured with the commercially available substrate D-galactose-1-phosphate (Gal-1-P). This is not the natural substrate of homologous nucleotidyltransferases, which is D-glucose-1-phosphate. Nucleotidyltransferase-S was incubated with 7 mM Gal-1-P, 7 mM dTTP, and 0.1 U of pyrophosphatase. The reaction was monitored by two different methods. The first was by TLC as shown in FIG. 5-2, which clearly showed the disappearance of dTTP and Gal-1-P and the formation of a new product with UV activity (dTDP-D-galactose). The second method of assay was a malachite green based inorganic phosphate assay. When dTTP is coupled to a Sugar-1-phosphate it releases pyrophosphate which is broken down to pyrophosphatase to 2 molecules of inorganic phosphate. In a system that is initially low in phosphate, this release of phosphate can be followed very sensitively by this assay as displayed on the right of FIG. 5-2 with a enzyme free negative control. Both assays clearly exhibited that the nucleotidyltransferase-S is active with the unnatural substrate D-galactose-1-phosphate. Thus we demonstrated we could couple the two enzyme reactions sequentially.

### Example 6

### One-Pot Coupling of Sugar-1-kinase-Nucleotidyltransferase Enzyme Activities

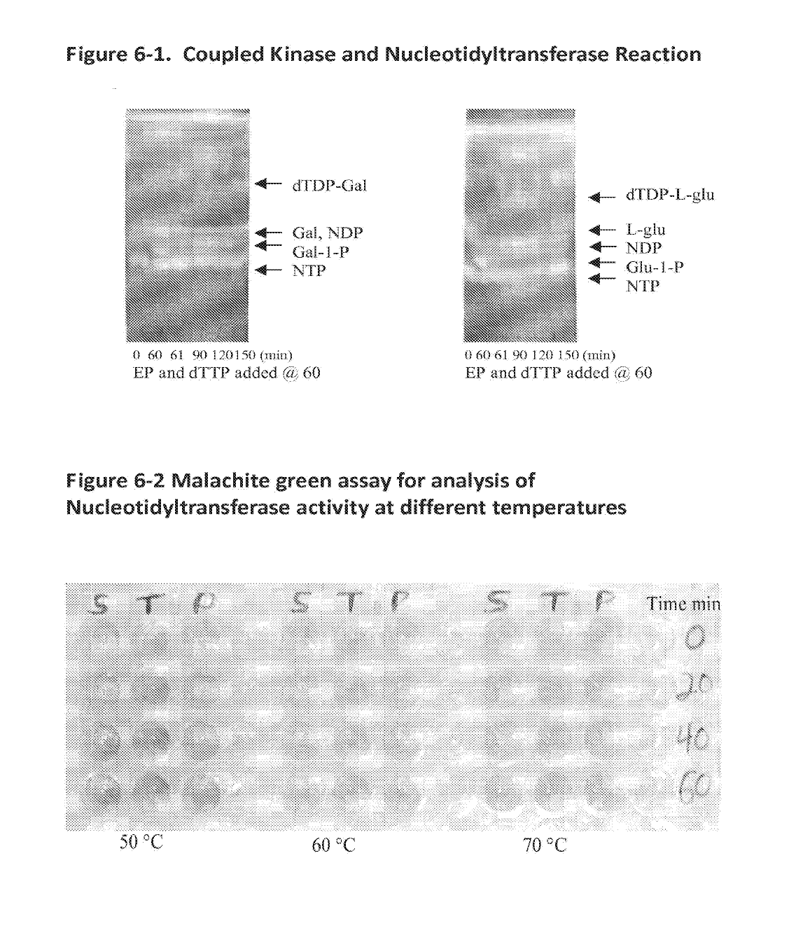

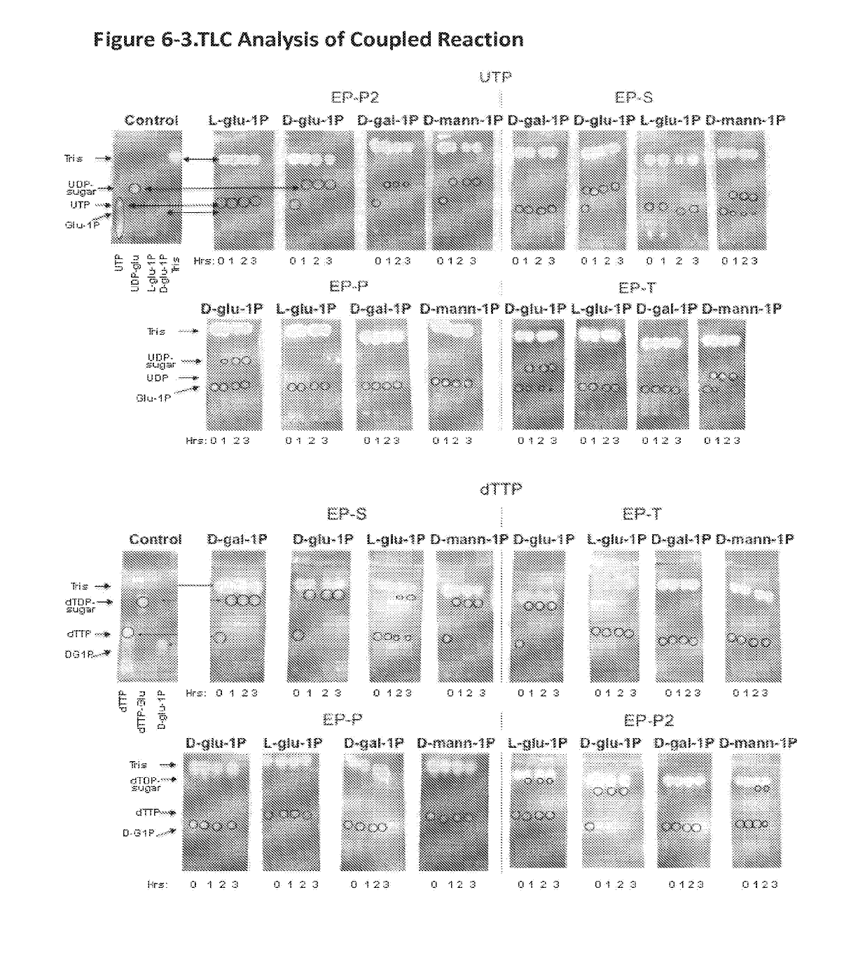

Initial coupling of the reaction was tested for 1-pot synthesis of NDP-sugars. The reaction was started with 12 mM ATP, 3.5 mM MgCl2, and 8 mM of either L-Glucose or D-galactose. Partially purified Sugar-1-kinase-P was added to the mixture and a sample was taken at 0 and 60 minutes. After 60 minutes, dTTP or UTP (8 mM), 20 μL nucleotidyltransferase-S, and 2 μL of commercially available thermostable pyrophosphatase were added to the reaction and samples were taken at different time points and analyzed by TLC as shown in FIG. 7-1. In the first 60 minutes both D-galactose and L-glucose were completely converted D-galactose-1-phosphate and L-glucose-1-phosphate respectively as determined by DNS assay and the appearance of a new spot on the TLC plate corresponding to an authentic standard of D-galactose-1-phosphate. Upon addition of the second enzyme and nucleotide, the formation of dTDP-D-galactose and dTDP-L-glucose began. The sugar nucleotides were more clearly visualized by UV, but can also been seen in the KMnO₄ stained TLC plates in FIG. 6-1. Additionally, the spot corresponding to Galactose-1-phosphate was gradually reduced in intensity.

Based on this data, we were capable of coupling the reaction of thermophilic nucleotidyltransferase and the mutant thermophilic sugar-1-kinase using the substrates D-galactose and dTTP. The conversion is estimated to be greater than 80% based on the loss of Gal-1-P and appearance of dTDP-Gal on TLC. The reaction with L-glucose and dTTP was also successful, however, the conversion was lower and estimated to be 20% by TLC. Testing UTP as an alternative nucleotide donor did not result in a successfully coupled reaction.

This reaction was optimized in terms of temperature for the nucleotidyltransferase step using the malachite green assay described in Example 1 for the release of phosphate. Partially purified cell extract was cleaned up by mini-gel filtration and mixed with D-Gal-1-P (15 mM) and dTTP (15 mM). The reactions were incubated at three different temperatures: 50° C., 60° C. and 70° C. Samples were taken at different times and analyzed. As exhibited in FIG. 6-2, nucleotidyltransferase-S was the most active and had best activity at 50° C. which was consistent with this enzyme being expressed the best in E. coli.

A fourth nucleotidlylytransferaseenzyme has been cloned from P. furiousus (EP-P2) that has previously been shown capable of converting the only commercially available L-sugar-1-phosphate (L-fucose-1-P),[47] transferring 82% to produce UDP-L-Fucose as determined by ESI-MS. EP-P2 additionally has a broad activity range on 6 other D-sugar-1-phosphates. [47] This enzyme was cloned as a His-tag fusion and purified by IMAC. Since we had 4 different enzymes (EP-S, EP-T, EP-P, and EP-P2) with different characteristics and substrate specificities, experiments were designed to test the substrate specificities on purified nucleotidlylytransferase enzyme and pure substrates. Reactions with each of the 4 nucleotidlylytransferase enzymes were set up using 4 different sugar-1-phosphates and 2 different nucleotides (32 reactions total), with each enzyme incubated near its optimal temperature. In a total volume of 200 μL the reactions contained 25 μL of purified enzyme, 5 mM MgCl₂, 6 mM nucleotide, 6 mM sugar-1-phosphate, 4 U thermophilic pyrophosphatase (commercially available). The 4 sugar-1-phosphates were D-glucose-1-phosphate, L-glucose-1-phosphate, D-galactose-1-phosphate, and D-mannose-1-phosphate, while the 2 nucleotides chosen were dTTP and UTP. EP-P and EP-P2 were incubated at 90° C., EP-T at 65° C., and EP-S at 45° C. Samples were taken at the time of enzyme addition and every hour for three hours and then analyzed by TLC using 80% aqueous acetonitrile +10 mM TBAHS; visualized by UV and stained in KMnO₄. The results are displayed below in FIG. 6-3.

The UV visible spots were circled in black for FIG. 6-3 to aid in visualization as only the nucleotides and nucleotide-activated sugars are UV active. The upper left plates show TLC of standard compounds. Using dTTP as a nucleotide EP-S displayed activity on all 4 tested substrates, EP-T converted D-glu-1P only, EP-P showed little to no activity, and EP-P2 showed activity on D-glu-1P, L-glu-1P, and D-mannose-1P. To our knowledge these are the first examples of commercially viable dTDP-L-glucose enzymatic production. Using UTP as the nucleotide substrate, EP-P2 displayed activity on all of the D-sugar-1P, but did not appear to appreciably convert L-glucose-1P. EP-S had good activity on both D-glu-1P and D-mann-1P. EP-P and EP-T both were only active on D-glucose-1P with UTP as the nucleotide. The results presented here are very promising and suggest that several of our cloned nucleotidlylytransferase enzymes are very capable, especially EP-S and EP-P2. Furthermore, many of the reactions proceeded to completion by the first time point analyzed.

### Example 7

### Further Relaxation of Substrate Specificity of Nucleotidyltransferase

Several mutants have been discovered previously that partially relax the specificity of the nucleotidyltransferase enzyme from Salmonella enterica. [27,31]. This information can be used to semi-rationally engineer the themostable nucleotidyltransferase-S for improved production of NDP-L-sugars. Any homologous site of mutation of thermostable nucleotidyltransferase enzymes will be targeted. These sites will be randomly mutagenized by incorporation of the degenerate codon NNS at the corresponding genetic loci. Additionally site for targeted saturation mutagenesis will be identified by homology modeling and analysis of the active site structure. The resulting mutants from saturation mutagenesis can be screened using the malachite green assay and TLC methods described in Example 1. Mutants identified with activity on desired substrates that is greater than wild-type activity will be carried on for additional rounds of mutagenesis and screening, until the desired level of activity is achieved or no further beneficial mutants can be identified. The new mutants will have the desired thermostability as well as high activity on a broad range of L- and D-sugar-1-phosphates.

### REFERENCES

1. Kren, V., Chemical biology and biomedicine of glycosylated natural compounds. Glycoscience Chemistry and Chemical Biology, ed. B. Fraser-Reid, K. Tatsuta, and J. Theim. Vol. III. 2001.
2. Lindhorst, T. K., Antitumor and antimicrobial glycoconjugates. Glycoscience Chemistry and Chemical Biology, ed. B. Fraser-Reid, K. Tatsuta, and J. Theim. Vol. III. 2001.
3. Thorson, J. S., et al., Natures Carbohydrate Chemists The Enzymatic Glycosylation of Bioactive Bacterial Metabolites. Current Organic Chemistry, 2001. 5(2): p. 139.
4. Thorson, J. S. and T. Vogt, Glycosylated Natural Products. Carbohydrate-based Drug Discovery, ed. C.-H. Wong. Vol. 2. 2003: Wiley. 685-711.
5. Weymouth-Wilson, A. C., The role of carbohydrates in biologically active natural products. Nat Prod Rep, 1997. 14(2): p. 99-110.
6. He, X., G. Agnihotri, and H. w. Liu, Novel Enzymatic Mechanisms in Carbohydrate Metabolism. Chem. Rev., 2000. 100(12): p. 4615-4662.
7 Liu, H. W. and J. S. Thorson, Pathways and mechanisms in the biogenesis of novel deoxysugars by bacteria. Annu Rev Microbiol, 1994. 48: p. 223-56.
8. Rupprath, C., T. Schumacher, and L. Elling, Nucleotide Deoxysugars: Essential Tools for the Glycosylation Engineering of Novel Bioactive Compounds. Current Medicinal Chemistry, 2005. 12(14): p. 1637.
9. Trefzer, A., A. Bechthold, and J. A. Salas, Genes and enzymes involved in deoxysugar biosynthesis in bacteria. Natural Product Reports, 1999. 16: p. 283-299.
10. Trefzer, A., et al., Rationally Designed Glycosylated Premithramycins: Hybrid Aromatic Polyketides Using Genes from Three Different Biosynthetic Pathways. Journal of the American Chemical Society, 2002. 124(21): p. 6056.
11. Borisova, S. A., et al., Biosynthesis of Desosamine: Construction of a New Macrolide Carrying a Genetically Designed Sugar Moiety. Org. Lett., 1999. 1(1): p. 133-136.
12. Zhao, L., et al., Engineering a Methymycin/Pikromycin-Calicheamicin Hybrid: Construction of Two New Macrolides Carrying a Designed Sugar Moiety. J. Am. Chem. Soc., 1999. 121(42): p. 9881-9882.
13. Zhao, L., et al., Mechanistic Studies of Desosamine Biosynthesis: C-4 Deoxygenation Precedes C-3 Transamination. J. Am. Chem. Soc., 1998. 120(46): p. 12159-12160.
14. Zhao, L., D. H. Sherman, and H. w. Liu, Biosynthesis of Desosamine: Construction of a New Methymycin/Neomethymycin Analogue by Deletion of a Desosamine Biosynthetic Gene. J. Am. Chem. Soc., 1998. 120(39): p. 10256-10257.
15. Ohuchi, T., et al., Cloning and expression of a gene encoding N-glycosyltransferase (ngt) from Saccarothrix aerocolonigenes ATCC39243. J Antibiot (Tokyo), 2000. 53(4): p. 393-403.
16. Sanchez, C., et al., The Biosynthetic Gene Cluster for the Antitumor Rebeccamycin: Characterization and Generation of Indolocarbazole Derivatives. Chemistry & Biology, 2002. 9(4): p. 519.
17. Zhang, C., et al., The in Vitro Characterization of the Iterative Avermectin Glycosyltransferase AveBI Reveals Reaction Reversibility and Sugar Nucleotide Flexibility. 2006. p. 16420-16421.
18. Dey, P. M., Galactokinase of Vicia faba seeds. Eur J Biochem, 1983. 136(1): p. 155-9.
19. Lavine, J. E., et al., Purification and properties of galactokinase from Tetrahymena thermophila. Biochim Biophys Acta, 1982. 717(1): p. 76-85.
20. Yang, J., et al., Studies on the substrate specificity of Escherichia coli galactokinase. Org Lett, 2003. 5(13): p. 2223-6.
21. Johnson, L. N. and D. Barford, Glycogen phosphorylase. The structural basis of the allosteric response and comparison with other allosteric proteins. J Biol Chem, 1990. 265(5): p. 2409-12.
22. Park, S. H., et al., Purification to apparent homogeneity and properties of pig kidney L-fucose kinase. J Biol Chem, 1998. 273(10): p. 5685-91.
23. Hoffmeister, D. and J. S. Thorson, Mechanistic implications of Escherichia coli galactokinase structure-based engineering. Chembiochem, 2004. 5(7): p. 989-92.
24. Yang, J., et al., Structure-based engineering of E. coli galactokinase as a first step toward in vivo glycorandomization. Chem Biol, 2005. 12(6): p. 657-64.
25. Yang, J., L. Liu, and J. S. Thorson, Structure-based enhancement of the first anomeric glucokinase. Chembiochem, 2004. 5(7): p. 992-6.
26. Hyun, C. G., et al., The biosynthesis of indolocarbazoles in a heterologous E. coli host. Chembiochem, 2003. 4(1): p. 114-7.
27. Hoffmeister, D., et al., Creation of the first anomeric D/L-sugar kinase by means of directed evolution. Proc Natl Acad Sci U.S.A, 2003. 100(23): p. 13184-9.
28. Lindquist, L., et al., Purification, characterization and HPLC assay of Salmonella glucose-1-phosphate thymidylyl-transferase from the cloned rfbA gene. Eur J Biochem, 1993. 211(3): p. 763-70.
29. Barton, W. A., et al., Structure, mechanism and engineering of a nucleotidylyltransferase as a first step toward glycorandomization. Nat Struct Biol, 2001. 8(6): p. 545-51.
30. Jiang, J., C. Albermann, and J. S. Thorson, Application of the nucleotidylyltransferase Ep toward the chemoenzymatic synthesis of dTDP-desosamine analogues. Chembiochem, 2003. 4(5): p. 443-6.
31. Jiang, J., J. B. Biggins, and J. S. Thorson, A General Enzymatic Method for the Synthesis of Natural and “Unnatural” UDP- and TDP-Nucleotide Sugars. J. Am. Chem. Soc., 2000. 122(28): p. 6803-6804.
32. Jiang, J., J. B. Biggins, and J. S. Thorson, Expanding the Pyrimidine Diphosphosugar Repertoire: The Chemoenzymatic Synthesis of Amino- and Acetamidoglucopyranosyl Derivatives This contribution was supported by the National Institutes of Health (GM58196 and CA84374), a Cancer Center Support Grant (CA-08748), and a grant from the Special Projects Committee of the Society of Memorial Sloan-Kettering Cancer Center. J. S. T. is an Alfred P. Sloan Research Fellow and a Rita Allen Foundation Scholar. Angew Chem Int Ed Engl, 2001. 40(8): p. 1502-1505.
33. Thorson, J. S., et al., Structure-based enzyme engineering and its impact on in vitro glycorandomization. Chembiochem, 2004. 5(1): p. 16-25.
34. Barton, W. A., et al., Expanding pyrimidine diphosphosugar libraries via structure-based nucleotidylyltransferase engineering. Proceedings of the National Academy of Sciences, 2002. 99(21): p. 13397.
35. Fu, X., et al., Antibiotic optimization via in vitro glycorandomization. Nat Biotechnol, 2003. 21(12): p. 1467-9.
36. Losey, H. C., et al., Incorporation of glucose analogs by GtfE and GtfD from the vancomycin biosynthetic pathway to generate variant glycopeptides. Chem Biol, 2002. 9(12): p. 1305-14.
37. Yang, J., et al., Natural product glycorandomization. Bioorg Med Chem, 2004. 12(7): p. 1577-84.
38. Nicolaou, K. C., et al., Solid- and solution-phase synthesis of vancomycin and vancomycin analogues with activity against vancomycin-resistant bacteria. Chemistry, 2001. 7(17): p. 3798-823.
39. Kopp, M., et al., SorF: a glycosyltransferase with promiscuous donor substrate specificity in vitro. Chembiochem, 2007. 8(7): p. 813-9.
40. Oberthur, M., et al., A systematic investigation of the synthetic utility of glycopeptide glycosyltransferases. J Am Chem Soc, 2005. 127(30): p. 10747-52.
41. Zhang, C., et al., Exploiting the reversibility of natural product glycosyltransferase-catalyzed reactions. Science, 2006. 313(5791): p. 1291-4.
42. Blanco, G., et al., Identification of a sugar flexible glycosyltransferase from Streptomyces olivaceus, the producer of the antitumor polyketide elloramycin. Chem Biol, 2001. 8(3): p. 253-63.
43. Menendez, N., et al., Deoxysugar transfer during chromomycin A3 biosynthesis in Streptomyces griseus subsp. griseus: new derivatives with antitumor activity. Appl Environ Microbiol, 2006. 72(1): p. 167-77.
44. Salas, A.P., et al., Deciphering the late steps in the biosynthesis of the anti-tumour indolocarbazole staurosporine: sugar donor substrate flexibility of the StaG glycosyltransferase. Mol Microbiol, 2005. 58(1): p. 17-27.
45. Fischer, C., et al., Digitoxosyltetracenomycin C and glucosyltetracenomycin C, two novel elloramycin analogues obtained by exploring the sugar donor substrate specificity of glycosyltransferase EImGT. J Nat Prod, 2002. 65(11): p. 1685-9.
46. Persson, M. and M. M. Palcic, A high-throughput pH indicator assay for screening glycosyltransferase saturation mutagenesis libraries. Anal Biochem, 2008. 378(1): p. 1-7.
47. Mizanur, R. M., C. J. Zea, and N. L. Pohl, Unusually broad substrate tolerance of a heat-stable archaeal sugar nucleotidyltransferase for the synthesis of sugar nucleotides. J Am Chem Soc, 2004. 126(49): p. 15993-8.
48. Thorson Chembiochem (2004) 5:992-6
49. Thorson et al. PNAS Oct. 15, 2002 vol. 99 no. 21 13397-13402

We claim:

1. An isolated, mutant sugar-1-kinase, wherein the isolated sugar-1-kinase has sugar-1-kinase activity in a sugar-1-kinase assay, and wherein the sugar-1-kinase comprises SEQ ID NO:10 and further comprises the following mutations:
(i) I312T, L332H, Y341P or Y341M, and F342K or F342T;
(ii) I312T and L332H;
(iii) Y341P or Y341M and F342K or F342T;
(iv) I312T, L332H, and Y341P or Y341M; or
(v) I312T, L332H, and F342K or F342T.

2. The isolated sugar-1-kinase of claim 1, wherein the sugar-1-kinase assay is a 3,5-dinitrosalicylic acid (DNS) assay, a thin layer chromatography assay or a high-performance liquid chromatography assay.

3. A polynucleotide encoding the sugar-1-kinase of claim 1.

4. An expression vector that comprises the polynucleotide of claim 3.

5. A host cell that comprises the sugar-1-kinase polynucleotide of claim 3.

6. A method of phosphorylating one or more sugars comprising contacting the sugars with the sugar-1-kinase of claim 1, wherein phosphorylated sugar-1-phosphates are produced.

7. The method of claim 6, wherein the reaction temperature is greater than 30° C. and the conversion rate of sugar to sugar-1-phosphate is greater than 50%.

8. The method of claim 6, wherein the sugar is an L-sugar or a D-sugar.

9. The method of claim 6, wherein the sugar is D-galactose, L-galactose, L-glucose, D-glucose, D-glucoronate, L-rhamnose, D-arabinose, L-arabinose, L-xylose, D-xylose, L-ribose, D-ribose, D-fucose, D-fucose, L-fucose, L-xylose, L-Ixyose, D-xylose, L-mannose, D-mannose, L-gulose, 6-azido-D-galactose, or a combination thereof.

10. The method of claim 6, further comprising contacting the sugar-1-phosphates with a nucleotidyltransferase to produce nucleoside-diphosphate (NDP) sugars.

11. The method of claim 10, wherein the nucleotidyltransferase and the sugar-1-kinase are contacted with the sugars at the same time or sequentially.
